(19) United States
(12) Patent Application Publication
(10) Pub. No.: US 2010/0095402 A1
(43) Pub. Date: Apr. 15, 2010

(54) Materials and methods for engineering resistance to tomato yellow leaf curl virus (tylcv) in plants,

(76) Inventors: Jane E. Polston, Gainesville, FL (US); Ernest Hiebert, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 11/919,106

(22) PCT Filed: Apr. 24, 2006
(86) PCT No.: PCT/US2006/015277

§ 371 Date: Dec. 21, 2009

Related U.S. Application Data
(60) Provisional application No. 60/673,934, filed on Apr. 22, 2005.

(51) Int. Cl. C12N 15/34; C12N 15/82; C12N 5/10; A01H 5/00

(52) U.S. Cl. 800/279; 536/23.72; 435/320.1; 435/418; 800/301

(57) ABSTRACT

The subject invention concerns materials and methods for providing genetically-engineered resistance in plants to geminivirus, such as, using polynucleotides containing all or a portion of a replication associated protein (Rep) gene of TYLCV and all or a portion of a Rep intergenic region (IR). Virus-resistant plants produced according to the present invention have horticulturally acceptable phenotypic traits. Methods of the invention comprise transforming a plant with a polynucleotide wherein when the polynucleotide is expressed in the plant, the transformed plant exhibits resistance to plant viral infections. An exemplified embodiment utilizes a polynucleotide comprising a Rep gene derived from a Florida isolate of TYLCV. The methods of the invention can be used to provide resistance to TYLCV infection in plants such as tomato and tobacco. The present invention also concerns transformed and transgenic plants and plant tissue that comprise a polynucleotide of the invention.

### CROSS-REFERENCE TO A RELATED APPLICATION

This application claims the benefit of U.S. Provisional Application Ser. No. 60/673,934, filed Apr. 22, 2005, which is hereby incorporated by reference herein in its entirety, including any figures, tables, nucleic acid sequences, amino acid sequences, and drawings.

The subject invention was made with government support under a research project supported by the U.S. Department of Agriculture-Tropical/Subtropical Agriculture Research Program, Grant No. 98341356784. The government has certain rights in the invention.

### BACKGROUND OF THE INVENTION

Tomato yellow leaf curl virus (TYLCV) belongs to the genus Begomovirus (Family: Geminiviridae). Begomoviruses are small, circular, single-stranded DNA plant viruses that are transmitted by the whitefly Bemisia tabaci (Genn.). TYLCV was first described in what is now Israel, and it is one of the most devastating viruses that can infect tomato (Cohen et al., 1966; Cohen et al. 1994. It now occurs in several locations around the world and has infected tomato in Florida since 1997 (Polston et al., 1997). Management of TYLCV in tomato production is very difficult, expensive, and has limited options. Host resistance to TYLCV has been difficult to find and develop (Lapidot et al., 2002). TYLCV-resistant tomato lines have been developed in Israel and in the United States over the last 20 and 12 years, respectively, using genes derived from wild species of Lycopersicon (Friedmann et al., 1998; Lapidot et al., 1997; Pilowsky et al., 1990; Scott et al., 1995; Zamir et al., 1994). However, the resistance is often unsatisfactory due to a linkage with poor fruit quality. Tolerant commercial cultivars collapse under early or severe infection pressure and require protection during early growth stages. These cultivars produce a yield in the presence of TYLCV, but still support the replication of TYLCV and can act as sources of TYLCV for susceptible crops (Gilreath et al.; Lapidot et al., 2001).

Several studies for genetically-engineered resistance to begomoviruses have focused on pathogen-derived resistance using partial, entire or mutated Begomovirus replication-associated protein (Rep) sequences. A Tomato golden mosaic virus (TGMV) Rep construct that contained 15 nucleotides upstream of the translation start codon, and plus 185 nucleotide downstream of the Rep 3′ end (including part of the REn and TrAP genes) was transformed into tobacco (Nicotiana tabacum) (Day et al., 1991). Transformed plants containing the construct in the sense orientation could not be obtained. Symptom expression was reduced and viral DNA replication was blocked by the antisense of this construct. Furthermore, levels of antisense RNA were correlated with symptom development (resistance). Resistance to the monopartite Tomato yellow leaf curl Sardinia virus (TYLCSV) was produced in N. benthamiana plants using the TYLCSV Rep gene with a deletion of 420 nucleotides (coding for 140 amino acid residues) from the 3′ end (Noris et al., 1996). However, resistance was transitory. Tomato plants were transformed with the same construct and it was found that accumulation of high levels of the truncated Rep protein was required for resistance, that this accumulation resulted in a curled phenotype, and that the resistance did not extend to an unrelated begomovirus. Bedahmane and Gronenborn (1997) tested transgenic tobacco expressing an antisense, truncated TYLCSV Rep (63-nucleotide leader and 288-nucleotide (5′) Rep sequences) transcript for virus resistance by agroinoculation. They found some resistant, symptomless lines with suppressed TYLCSV replication as well as some tolerant lines with reduced TYLCSV replication. Transcript analysis indicated that plants with elevated transgene transcript levels consistently exhibited a higher degree of virus resistance. Polston and Hiebert (2001) transformed tomato with Tomato mottle virus (ToMoV) Rep gene (sense orientation), including the upstream 113 nucleotide of the intergenic region (IR), and demonstrated that a single copy of the transgene in tomato provided stable and high levels of resistance to ToMoV under field conditions (evaluated in the R₁ through R₅ generation). Both tolerance and immunity to ToMoV were seen in transformed plants. Yields of transformed plants from the R₄ generation were found to be equal to or higher than those of the untransformed parent in the absence of virus, and more than twice as great in the presence of ToMoV.

Noncoding begomovirus sequences OR) also have been tested for the generation engineered virus resistance. Pooggin et al. (2003) demonstrated that transient expression of both sense and antisense Vigna mungo yellow mosaic virus (VMYMV) promoter sequences in the IR resulted in complete recovery in infected VMYMV plants. The recovery of the whole plant from VMYMV indicated that the interfering signal spread throughout the plant. They proposed that RNA interference (post transcriptional gene silencing (PTGS)) as has been described for RNA viruses also was possible for a DNA virus.

### BRIEF SUMMARY OF THE INVENTION

The subject invention concerns materials and methods for providing broad resistance to tomato yellow leaf curl geminivirus in plants while maintaining acceptable phenotypic characteristics of the plant. In an exemplified embodiment, the subject invention provides resistance to TYLCV in tomato. In one embodiment, TYLCV resistance is provided by transforming a plant with a polynucleotide wherein the polynucleotide comprise a truncated sense or antisense version of the replication associated protein (Rep) gene of TYLCV. Exemplified herein is the use of a Rep gene from TYLCV (Florida isolate). Preferably, the polynucleotide also comprises all or part of a Rep intergenic region (IR) that is 5′ (i.e., upstream of) to the Rep gene transcript start site.

### BRIEF DESCRIPTION OF THE DRAWINGS

FIGS. 1A and 1B show a diagram demonstrating the development of TYLCV gene expression constructs engineered into pKYLX71:35S² vector or non-enhanced promoter vector. FIG. 1A shows gene fragment containing the full length sequence of the TYLCV Rep gene was cloned directly into pKYLX:35S². FIG. 1B shows a modified (non-enhanced 35S promoter) binary vector pKYLX into which the full-length sequence of the TYLCV Rep gene was cloned. In the generic construct, LB=left border and RB=right border of the T-DNA; 35S²=modified CaMV 35S promoter with a duplicated “enhancer” region; TYLCV insert=insertion site of the various TYLCV constructs; RBCS Ter=rbcS-terminator; and NPT II=kanamycin resistance gene. In the construct names, T stands for “truncated”; Δ stands for antisense. TYLCV sequence GenBank accession no. AY530931. Capital letters are carryovers from restriction sites used in construction. Constructs are not to scale.

FIG. 2 shows a phenotype of the R₁-generation tomato plants being screened for resistance to TYLCV. Plant on left is a non-transformed parent (Fla. 7613). Plant on the right is transformed with pKY2/5Rep.

FIGS. 3A and 3B are transgenic tomato showing resistance in the field. Tomatoes inoculated with TYLCV and transplanted in the field. FIG. 3A is a non-transformed plant of Fla. 7613. FIG. 3B is an R₂ generation plant transformed with pKY2/5 Rep (Fla. 7613 genetic background). Picture was taken approximately 60 days after transplanting.

### BRIEF DESCRIPTION OF THE SEQUENCES

SEQ ID NO: 1 is a PCR primer which can be used in the subject invention.

SEQ ID NO: 2 is a PCR primer which can be used in the subject invention.

SEQ ID NO: 3 is a PCR primer which can be used in the subject invention.

SEQ ID NO: 4 is a PCR primer which can be used in the subject invention.

SEQ ID NO: 5 is a PCR primer which can be used in the subject invention.

SEQ ID NO: 6 is a PCR primer which can be used in the subject invention.

SEQ ID NO: 7 is a PCR primer which can be used in the subject invention.

SEQ ID NO: 8 is a PCR primer which can be used in the subject invention.

SEQ ID NO: 9 is a PCR primer which can be used in the subject invention.

SEQ ID NO: 10 is a PCR primer which can be used in the subject invention.

SEQ ID NO: 11 is a PCR primer which can be used in the subject invention.

SEQ ID NO: 12 is a PCR primer which can be used in the subject invention.

SEQ ID NO: 13 is a PCR primer which can be used in the subject invention.

SEQ ID NO: 14 is a PCR primer which can be used in the subject invention.

SEQ ID NO: 15 is a PCR primer which can be used in the subject invention.

SEQ ID NO: 16 is a polynucleotide which can be used according to the subject invention.

SEQ ID NO: 17 is a polynucleotide which can be used according to the subject invention.

SEQ ID NO: 18 is a polynucleotide which can be used according to the subject invention.

SEQ ID NO: 19 is a polynucleotide which can be used according to the subject invention.

SEQ ID NO: 20 is a polynucleotide which can be used according to the subject invention.

SEQ ID NO: 21 is a polynucleotide which can be used according to the subject invention.

SEQ ID NO: 22 is a polynucleotide which can be used according to the subject invention.

SEQ ID NO: 23 is a polynucleotide which can be used according to the subject invention.

### DETAILED DISCLOSURE OF THE INVENTION

The present invention provides a means for producing geminivirus resistance in tomato and other crops that are susceptible to infection by geminivirus. In one aspect, the subject invention provides resistance to Tomato yellow leaf curl virus (TYLCV). In an exemplified embodiment, plants having resistance to TYLCV-Is (tomato yellow leaf curl virus-Israel) were produced. The transformed and transgenic plants produced according to the present invention exhibit normal phenotypic qualities such that the plants and their produced crop are acceptable for commercial use. Development of TYLCV resistance through conventional breeding has been slow (more than 10 years for traditional breeding versus 3-5 years for genetic engineered technology), usually depends upon multiple genes, and often is compromised by linkages of resistance genes to undesirable horticultural characteristics. The present invention greatly simplifies production of geminivirus resistant plants and the introduction of a TYLCV resistance gene into desirable tomato lines and other plants.

The subject invention concerns methods for providing resistance to infection by a geminivirus in a plant or plant tissue by transforming a plant or plant tissue with a polynucleotide comprising a portion of a TYLCV Rep gene. In one embodiment, the method provides resistance to infection by a Tomato yellow leaf curl virus. Resistance to TYLCV strains specifically contemplated by the subject methods includes, but is not limited to, TYLCV, TYLCV-Mld (Tomato yellow leaf curl virus-Mild), TYLCV-IR (Tomato yellow leaf curl virus-Iran), and TYLCV-SD (Tomato yellow leaf curl virus-Sudan). Resistance to TYLCV isolates specifically contemplated by the subject methods, includes, but is not limited to, TYLCV-[Alm] (Tomato yellow leaf curl virus-[Almeria]), TYLCV-[Aic] (Tomato yellow leaf curl virus-[Aichi]), TYLCV-[CU] (Tomato yellow leaf curl virus-[Cuba]), TYLCV-[DO] (Tomato yellow leaf curl virus-[Dominican Republic]), TYLCV-[Fl] (Tomato yellow leaf curl virus-[Florida]), TYLCV-[PT] (Tomato yellow leaf curl virus-[Portugal]), TYLCV-[SA] (Tomato yellow leaf curl virus-[Saudi Arabia]), TYLCV-[Shi] (Tomato yellow leaf curl virus-[Shizuokua]), and TYLCV-[ES7297] (Tomato yellow leaf curl virus-[Spain7297]). Plants that can be provided with resistance to TYLCV infection include, but are not limited to tomato, tobacco, pepper, bean, statice, petunia, lisianthus, tomatillo, as well as other plants susceptible to infection by TYLCV.

In an exemplified embodiment, the subject invention provides plants having genetically-engineered resistance to TYLCV using a polynucleotide which comprises a Rep gene nucleotide sequence that encodes all or a portion of the replication associated (Rep) protein of TYLCV and all or a portion of the intergenic region (1R) of a TYLCV. Thus, the subject invention concerns polynucleotides that when provided in a plant confer resistance to TYLCV infection to the plant. The Rep sequences used in the present invention can be from any strain of TYLCV, including, but not limited to, TYLCV, TYLCV-Mld, TYLCV-IR, and TYLCV-SD. The Rep sequences used in the present invention can also be from any isolate of TYLCV, including, but not limited to, TYLCV-[Alm], TYLCV-[Aic], TYLCV-[CU], TYLCV-[DO], TYLCV-[Fl], TYLCV-[PT], TYLCV-[SA], TYLCV-[Shi], and TYLCV-[ES7297]. In one embodiment, the Rep gene sequence of a polynucleotide of the present invention is from TYLCV-[Florida] (TYLCV-Fl). In one embodiment, a polynucleotide of the invention comprises from about 50 to 100 nucleotides of a TYLCV intergenic region and about 300 to 700 nucleotides of the 5′ terminus of a TYLCV Rep gene. In a further embodiment, a polynucleotide of the invention comprises about 70 to 90 nucleotides of the IR and about 400 to 450 nucleotides of the 5′ terminus of the Rep gene. In a still further embodiment, a polynucleotide of the invention comprises about 80 to 85 nucleotides of the IR and about 410 to about 430 nucleotides of the 5′ terminus of the Rep gene. In an exemplified embodiment, a polynucleotide of the invention has the nucleotide sequence shown in SEQ ID NO: 16.

In another embodiment, a polynucleotide of the invention comprises from about 50 to 100 nucleotides of a TYLCV IR and about 300 to 700 nucleotides of the 5′ terminus of a TYLCV Rep gene, wherein the IR and Rep sequences are in the antisense orientation. In a further embodiment, a polynucleotide of the invention comprises from about 80 to 90 nucleotides of a TYLCV IR and about 500 to 600 nucleotides of the 5′ terminus of the Rep gene, both in the antisense orientation. In an exemplified embodiment, a polynucleotide of the invention has the nucleotide sequence shown in SEQ ID NO: 17. As used herein, the term “antisense” refers to polynucleotides that provide for transcribed sequences that are at least partially complementary to the transcript from genes that are in the normal, sense orientation.

In a still further embodiment, a polynucleotide of the invention comprises, in a sense or antisense orientation, from about 50 to 100 nucleotides of a TYLCV IR, followed by a complete Rep gene sequence or a fragment thereof. In an exemplified embodiment, a polynucleotide of the invention has the nucleotide sequence shown in any of SEQ ID NO: 19, SEQ ID NO: 20, or SEQ ID NO: 21.

In a still further embodiment, a polynucleotide of the invention comprises a first polynucleotide that comprises all or a portion of a TYLCV IR, followed by all or a portion of the Rep gene, operatively linked to a second polynucleotide comprising, in antisense orientation, all or a portion of the Rep gene, followed by all or a portion of a TYLCV IR also in antisense orientation. Optionally, a linker nucleotide sequence can be provided operatively linking the polynucleotides. In one embodiment, the linker sequence can comprise a sequence that is downstream of the 3′ end of the Rep gene. In one embodiment, a polynucleotide of the invention comprises from about 50 to 100 nucleotides, or about 70 to 90 nucleotides, or about 80 to 85 nucleotides, of an IR, followed by a complete Rep gene sequence or a fragment thereof of about 300 to 700 nucleotides of the 5′ terminus of the Rep gene, optionally followed by about 10 to 100 nucleotides downstream of the 3′ end of a Rep gene, and/or optionally followed by a linker of between about 1 to 100 nucleotides, and/or optionally followed by a complete Rep gene in antisense orientation or about 300 to 700 nucleotides, or about 500 to 600 nucleotides, of the 5′ terminus of a Rep gene in the antisense orientation and about 50 to 100 nucleotides, or about 80 to 90 nucleotides, of a TYLCV IR also in the antisense orientation. In one embodiment, the polynucleotide of SEQ ID NO: 16 or SEQ ID NO: 19 is operatively linked to the polynucleotide of SEQ ID NO: 17. In an exemplified embodiment, a polynucleotide of the invention has the nucleotide sequence shown in SEQ ID NO: 18.

Also within the scope of the present invention are fragments and variants of the polynucleotides of the invention that can provide a plant with resistance to infection by TYLCV. Fragments and variants can be prepared and tested for ability to confer TYLCV resistance using standard methods known in the art.

Polynucleotides of the present invention can be introduced directly into plants, such as tomato, by Agrobacterium-mediated transformation, and transformed and transgenic plant lines prepared therefrom. Plants containing a polynucleotide of the invention can also be prepared through conventional breeding from a transformed or transgenic breeding line. Plants having a polynucleotide of the invention are protected from or less susceptible to TYLCV infection. Thus, the subject invention also concerns new breeding lines of plants, including tomato, with high levels of resistance to TYLCV, such as TYLCV-[Fl]. By using new breeding lines that comprise a polynucleotide of the present invention, breeders can generate new cultivars of tomato that are resistant to virus without sacrificing other desired agronomic and horticulture features. Preferably, polynucleotides of the present invention comprise markers to assist in breeding efforts (marker-assisted selection).

In one embodiment of the invention, a virus-resistant transgenic plant line prepared according to the methods described herein is crossed with a transgenic plant line that is resistant to the same virus and derived from a different transformation event to produce hybrids that exhibit increased virus resistance over the parent lines. These hybrid plants are within the scope of the present invention.

Because of the high levels of resistance conferred by the methods of the present invention, application of new tomato cultivars generated from breeding lines containing a polynucleotide of the present invention can greatly reduce the production loss caused by virus infection which can decrease fruit yield by up to 100%. Moreover, the use of cultivars comprising a polynucleotide of the present invention can significantly reduce the dependence on pesticides to control whitefly that is the vector of TYLCV and other geminiviruses, and consequently reduce tomato production costs and environmental contamination by pesticides. At this time, TYLCV management is heavily dependent upon a single insecticide that is translocated systemically in plants. Once the whiteflies become resistant to this insecticide, management of TYLCV will be nearly impossible. Therefore, application of the present invention can benefit tomato growers and society as a whole by increasing productivity and reducing production costs and environment contamination.

In contrast to the results obtained by Brunetti et al. (1997), the use of polynucleotides of the invention, to date, have not shown any adverse affects on the phenotype of virus-resistant but non-inoculated transformed plants. Resistant plants showed no evidence of virus replication 30 to 60 days after inoculation, as established by nucleic acid spot hybridization and PCR. A polynucleotide of the present invention can be readily moved either through transformation or through conventional breeding techniques into new horticultural backgrounds that will provide superior horticultural traits for production. In addition, a polynucleotide of the present invention in plants does not interfere with fruit size, as do the currently available resistance genes.

The subject invention also concerns polynucleotide expression constructs comprising a polynucleotide sequence of the present invention. Thus, the subject invention concerns expression constructs comprising a polynucleotide sequence comprising the nucleotide sequence shown in any of SEQ ID NOs: 16, 17, 18, 19, 20, 21, 22, or 23, or a functional fragment or variant of any of SEQ ID NOs: 16, 17, 18, 19, 20, 21, 22, or 23. In a preferred embodiment, an expression construct of the present invention provides for overexpression of an operably linked polynucleotide of the invention.

Expression constructs of the invention generally include regulatory elements that are functional in the intended host cell in which the expression construct is to be expressed. Thus, a person of ordinary skill in the art can select regulatory elements for use in plant host cells, bacterial host cells, yeast host cells, insect host cells, mammalian host cells, and human host cells. Regulatory elements include promoters, transcription termination sequences, translation termination sequences, enhancers, and polyadenylation elements. As used herein, the term “expression construct” refers to a combination of nucleic acid sequences that provides for transcription of an operably linked nucleic acid sequence. As used herein, the term “operably linked” refers to a juxtaposition of the components described wherein the components are in a relationship that permits them to function in their intended manner. In general, operably linked components are in contiguous relation.

An expression construct of the invention can comprise a promoter sequence operably linked to a polynucleotide sequence encoding a polypeptide of the invention. Promoters can be incorporated into a polynucleotide using standard techniques known in the art. Multiple copies of promoters or multiple promoters can be used in an expression construct of the invention. In a preferred embodiment, a promoter can be positioned about the same distance from the transcription start site in the expression construct as it is from the transcription start site in its natural genetic environment. Some variation in this distance is permitted without substantial decrease in promoter activity. A transcription start site is typically included in the expression construct. Preferably, the promoter associated with an expression construct of the invention provides for overexpression of an operably linked polynucleotide of the invention.

If the expression construct is to be provided in or introduced into a plant cell, then plant viral promoters, such as, for example, a cauliflower mosaic virus (CaMV) 35S (including the enhanced CaMV 35S² promoter (see, for example U.S. Pat. No. 5,106,739 and An, 1987)) or a CaMV 19S promoter can be used. Other promoters that can be used for expression constructs in plants include, for example, prolifera promoter, Ap3 promoter, heat shock promoters, T-DNA 1′- or 2′-promoter of A. tumefaciens, polygalacturonase promoter, chalcone synthase A (CHS-A) promoter from petunia, tobacco PR-1a promoter, ubiquitin promoter, actin promoter, alcA gene promoter, pint promoter (Xu et al., 1993), maize WipI promoter, maize trpA gene promoter (U.S. Pat. No. 5,625,136), maize CDPK gene promoter, alfalfa histone H3.2 promoter (Kelemen et al., 2002) and RUBISCO SSU promoter (U.S. Pat. No. 5,034,322) can also be used. Root-specific promoters, such as any of the promoter sequences described in U.S. Pat. No. 6,455,760 or U.S. Pat. No. 6,696,623, or in published U.S. patent application Nos. 20040078841; 20040067506; 20040019934; 20030177536; 20030084486; or 20040123349, can be used with an expression construct of the invention. Constitutive promoters (such as the CaMV, ubiquitin, actin, or NOS promoter), developmentally-regulated promoters, and inducible promoters (such as those promoters than can be induced by heat, light, hormones, or chemicals) are also contemplated for use with polynucleotide expression constructs of the invention.

Tissue-specific promoters, for example fruit-specific promoters, such as the E8 promoter of tomato (accession number: AF515784; Good et al. (1994)) can also be used. Flower organ-specific promoters can be used with an expression construct of the present invention for expressing a polynucleotide of the invention in the flower organ of a plant. Examples of flower organ-specific promoters include any of the promoter sequences described in U.S. Pat. Nos. 6,462,185; 5,639,948; and 5,589,610. Seed-specific promoters such as the promoter from a β-phaseolin gene (for example, of kidney bean) or a glycinin gene (for example, of soybean), and others, can also be used.

For expression in prokaryotic systems, an expression construct of the invention can comprise promoters such as, for example, alkaline phosphatase promoter, tryptophan (tip) promoter, lambda P_{L} promoter, β-lactamase promoter, lactose promoter, phoA promoter, T3 promoter, T7 promoter, or tac promoter (de Boer et al., 1983). Promoters suitable for use with an expression construct of the invention in yeast cells include, but are not limited to, 3-phosphoglycerate kinase promoter, glyceraldehyde-3-phosphate dehydrogenase promoter, metallothionein promoter, alcohol dehydrogenase-2 promoter, and hexokinase promoter.

Expression constructs of the invention may optionally contain a transcription termination sequence, a translation termination sequence, a sequence encoding a signal peptide, and/or enhancer elements. Transcription termination regions can typically be obtained from the 3′ untranslated region of a eukaryotic or viral gene sequence. Transcription termination sequences can be positioned downstream of a coding sequence to provide for efficient termination. Classical enhancers are cis-acting elements that increase gene transcription and can also be included in the expression construct. Classical enhancer elements are known in the art, and include, but are not limited to, the CaMV 35S enhancer element, cytomegalovirus (CMV) early promoter enhancer element, and the SV40 enhancer element. Intron-mediated enhancer elements that enhance gene expression are also known in the art. These elements must be present within the transcribed region and are orientation dependent. Examples include the maize shrunken-1 enhancer element (Clancy and Hannah, 2002).

DNA sequences which direct polyadenylation of mRNA transcribed from the expression construct can also be included in the expression construct, and include, but are not limited to, an octopine synthase or nopaline synthase signal. The expression constructs of the invention can also include a polynucleotide sequence that directs transposition of other genes, i.e., a transposon.

Expression constructs can also include one or more dominant selectable marker genes, including, for example, genes encoding antibiotic resistance and/or herbicide-resistance for selecting transformed cells. Antibiotic-resistance genes can provide for resistance to one or more of the following antibiotics: hygromycin, kanamycin, bleomycin, G418, streptomycin, paromomycin, neomycin, and spectinomycin. Kanamycin resistance can be provided by neomycin phosphotransferase (NPT II). In one embodiment, a polynucleotide encoding NPT II is incorporated into an expression construct of the invention. Herbicide-resistance genes can provide for resistance to phosphinothricin acetyltransferase, sulfonylurea, or glyphosate. Other markers used for cell transformation screening include genes encoding β-glucuronidase (GUS), β-galactosidase, luciferase, nopaline synthase, chloramphenicol acetyltransferase (CAT), green fluorescence protein (GFP), or enhanced GFP (Yang et al., 1996). Cells transformed with an expression construct of the invention containing an antibiotic or herbicide resistance marker gene can be identified by their ability to grow in the presence of the antibiotic or herbicide.

The subject invention also concerns polynucleotide vectors comprising a polynucleotide sequence of the invention that encodes a polypeptide of the invention. Unique restriction enzyme sites can be included at the 5′ and 3′ ends of an expression construct or polynucleotide of the invention to allow for insertion into a polynucleotide vector. As used herein, the term “vector” refers to any genetic element, including for example, plasmids, cosmids, chromosomes, phage, virus, and the like, which is capable of replication when associated with proper control elements and which can transfer polynucleotide sequences between cells. Vectors contain a nucleotide sequence that permits the vector to replicate in a selected host cell. A number of vectors are available for expression and/or cloning, and include, but are not limited to, pBR322, pUC series, pGEM series, M13 series, and pBLUESCRIPT vectors (Stratagene, La Jolla, Calif.).

The present invention also concerns cells infected, transformed, or transfected with a polynucleotide of the present invention. In one embodiment, the cell comprises a polynucleotide that comprises a complete or partial Rep gene of a TYLCV. In one embodiment, the Rep nucleotide sequences of the polynucleotide are derived from TYLCV-Fl. In an exemplified embodiment, the polynucleotide comprises a nucleotide sequence shown in SEQ ID NO: 16, SEQ ID NO: 19, or SEQ ID NO: 20. In another embodiment, the cell comprises a polynucleotide comprising a Rep antisense sequence. In an exemplified embodiment, the polynucleotide comprises a nucleotide sequence shown in SEQ ID NO: 17, SEQ ID NO: 21, or SEQ ID NO: 23. In a further embodiment, a polynucleotide of the invention comprises a full length Rep gene fused to an antisense orientation of the 5′ end of a Rep gene, thus combining Rep sequences of both sense and antisense orientation. In an exemplified embodiment, the polynucleotide comprises a nucleotide sequence shown in SEQ ID NO: 18. In one embodiment, the polynucleotide is incorporated into a suitable vector, and the recombinant vector is used to transform a bacterium or other host which can then be used to introduce the polynucleotide into a plant cell. Suitable hosts that can be infected, transformed, or transfected with a polynucleotide of the invention include gram positive and gram negative bacteria such as E. coli and Bacillus subtilis. Other suitable hosts include Agrobacterium cells, insect cells, plant cells, and yeast cells. Agrobacterium containing a polynucleotide of the invention can be used to transform plant cells with the polynucleotide according to standard methods known in the art. For Agrobacterium transformation, polynucleotide vectors of the invention can also include T-DNA sequences. Polynucleotides can also be introduced into plant cells by a biolistic method (Carrer, 1995), by electroporation, by direct gene injection, and by other methods known in the art. Plants can also be transformed with polynucleotides of the present invention using marker-free transformation techniques (Zuo et al., 2002).

The subject invention also concerns transformed and transgenic plants and plant tissue, including plant seeds, that comprise a polynucleotide of the present invention and that exhibit resistance to infection by plant geminiviruses such as TYLCV, including TYLCV-[FL]. In one embodiment, a transformed or transgenic plant of the invention comprises a polynucleotide that comprises a complete or partial Rep gene of a TYLCV. In one embodiment, the Rep gene used in the subject polynucleotides is derived from TYLCV-[Fl]. In an exemplified embodiment, the polynucleotide comprises a nucleotide sequence shown in SEQ ID NO: 16, SEQ ID NO: 19, or SEQ ID NO: 20. In those embodiments of polynucleotides of the invention, such as that shown in SEQ ID NO: 18, that comprise a full-length Rep gene operatively linked to the Δ2/5Rep polynucleotide construct, the transcript of the transgene can form a large hairpin structure (the antisense 2/5Rep transcript segment binding to sense transcript segment of the 5′ end of the full length Rep leaving a segment to form a large loop; the loop segment of the transcript of SEQ ID NO: 18 comprises nucleotides 1505 to 2020). Such hairpin structures are known to induce gene silencing (Smith et al., 2000) and virus resistance. In another embodiment, the plant comprises a polynucleotide comprising a Rep antisense sequence (i.e., a Rep sequence in antisense orientation relative to a Rep sense sequence). In an exemplified embodiment, the polynucleotide comprises a nucleotide sequence shown in SEQ ID NO: 17, SEQ ID NO: 21, or SEQ ID NO: 23. Transformed and transgenic plants and plant tissue of the invention can be prepared from plants such as tomato (Lycopersicon esqulentum), tobacco (Nicotiana species), statice (Limoniunz sinuatum), petunia (Petunia hybrida), lisianthus (Eustoma grandiflora), tomatillo (Physalis ixocarpa), pepper (Capsicum annuum), bean (Phaseolus vulgaris), and others that can be or that are susceptible to being infected by TYLCV. The subject invention also concerns progeny of transgenic plants of the invention.

As used herein, the terms “nucleic acid” and “polynucleotide” refer to a deoxyribonucleotide, ribonucleotide or a mixed deoxyribonucleotide and ribonucleotide polymer in either single- or double-stranded form, and unless otherwise limited, would encompass known analogs of natural nucleotides that can function in a similar manner as naturally-occurring nucleotides. The polynucleotide sequences include both the DNA strand sequence that is transcribed into RNA and the RNA sequence that is translated into protein. The complementary sequence of any nucleic acid or polynucleotide of the present invention is also contemplated within the scope of the invention. The polynucleotide sequences include both full-length sequences that encode the Rep protein of the invention as well as shorter sequences derived from the full-length sequences. It is understood that a particular polynucleotide sequence includes the degenerate codons of the native sequence or sequences which may be introduced to provide codon preference in a specific host cell.

Polynucleotides of the present invention can be composed of either RNA or DNA. Preferably, the polynucleotides are composed of DNA. The subject invention also encompasses those polynucleotides that are complementary in sequence to the polynucleotides disclosed herein. Polynucleotides and polypeptides of the invention can be provided in purified or isolated form.

Because of the degeneracy of the genetic code, a variety of different polynucleotide sequences can encode a polypeptide of the present invention. A table showing all possible triplet codons (and where U also stands for T) and the amino acid encoded by each codon is described in Lewin (1985). In addition, it is well within the skill of a person trained in the art to create alternative polynucleotide sequences encoding the same, or essentially the same, polypeptides of the subject invention. These degenerate variant and alternative polynucleotide sequences are within the scope of the subject invention. As used herein, references to “essentially the same” sequence refers to sequences which encode amino acid substitutions, deletions, additions, or insertions which do not materially alter the functional activity of the polypeptide encoded by the polynucleotides of the present invention.

Substitution of amino acids other than those specifically exemplified or naturally present in a polypeptide of the invention are also contemplated within the scope of the present invention. For example, non-natural amino acids can be substituted for the amino acids of a polypeptide, so long as the polypeptide having the substituted amino acids retains substantially the same functional activity as the polypeptide in which amino acids have not been substituted. Examples of non-natural amino acids include, but are not limited to, ornithine, citrulline, hydroxyproline, homoserine, phenylglycine, taurine; iodotyrosine, 2,4-diaminobutyric acid, α-amino isobutyric acid, 4-aminobutyric acid, 2-amino butyric acid, γ-amino butyric acid, ε-amino hexanoic acid, 6-amino hexanoic acid, 2-amino isobutyric acid, 3-amino propionic acid, norleucine, norvaline, sarcosine, homocitrulline, cysteic acid, τ-butylglycine, τ-butylalanine, phenylglycine, cyclohexylalanine, β-alanine, fluoro-amino acids, designer amino acids such as β-methyl amino acids, C-methyl amino acids, N-methyl amino acids, and amino acid analogues in general. Non-natural amino acids also include amino acids having derivatized side groups. Furthermore, any of the amino acids in the protein can be of the D (dextrorotary) form or L (levorotary) form. Allelic variants of a protein sequence of a polypeptide of the present invention are also encompassed within the scope of the invention.

Amino acids can be generally categorized in the following classes: non-polar, uncharged polar, basic, and acidic. Conservative substitutions whereby a polypeptide encoded by a polynucleotide of the present invention having an amino acid of one class is replaced with another amino acid of the same class fall within the scope of the subject invention so long as the polynucleotide encoding the substitution still retains substantially the same functional activity as the polynucleotide that does not have the substitution. Polynucleotides encoding a polypeptide having one or more amino acid substitutions in the sequence are contemplated within the scope of the present invention. Table 6 below provides a listing of examples of amino acids belonging to each class. Single letter amino acid abbreviations are defined in Table 7.

The subject invention also concerns variants of the polynucleotides of the present invention that encode polypeptides of the invention. Variant sequences include those sequences wherein one or more nucleotides of the sequence have been substituted, deleted, and/or inserted. The nucleotides that can be substituted for natural nucleotides of DNA have a base moiety that can include, but is not limited to, inosine, 5-fluorouracil, 5-bromouracil, hypoxanthine, 1-methylguanine, 5-methylcytosine, and tritylated bases. The sugar moiety of the nucleotide in a sequence can also be modified and includes, but is not limited to, arabinose, xylulose, and hexose. In addition, the adenine, cytosine, guanine, thymine, and uracil bases of the nucleotides can be modified with acetyl, methyl, and/or thio groups. Sequences containing nucleotide substitutions, deletions, and/or insertions can be prepared and tested using standard techniques known in the art.

Polynucleotides and proteins of the subject invention can also be defined in terms of more particular identity and/or similarity ranges with those exemplified herein. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence exemplified herein. Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

The subject invention also contemplates those polynucleotide molecules having sequences which are sufficiently homologous with the sequences exemplified herein so as to permit hybridization with that sequence under standard stringent conditions and standard methods (Maniatis, T. et al., 1982). As used herein, “stringent” conditions for hybridization refers to conditions wherein hybridization is typically carried out overnight at 20-25 C below the melting temperature (Tm) of the DNA hybrid in 6×SSPE, 5×Denhardt's solution, 0.1% SDS, 0.1 mg/ml denatured DNA. The melting temperature is described by the following formula (Beltz, G. A. et al., 1983):

Tm=81.5 C+16.6 Log[Na+]+0.41(% G+C)−0.61(% formamide)−600/length of duplex in base pairs.

Washes are typically carried out as follows:

(1) Twice at room temperature for 15 minutes in 1×SSPE, 0.1% SDS (low stringency wash).

(2) Once at Tm-20 C for 15 minutes in 0.2×SSPE, 0.1% SDS (moderate stringency wash).

Thus, specifically contemplated within the scope of the invention are polynucleotide sequences that hybridize under stringent conditions with the sequence of SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 23.

### Materials and Methods

### Construction of Transgene Expression Vectors.

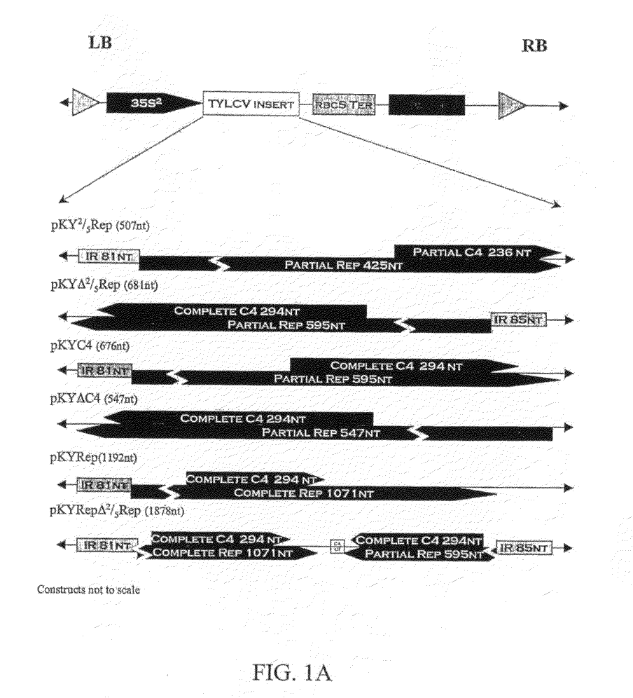

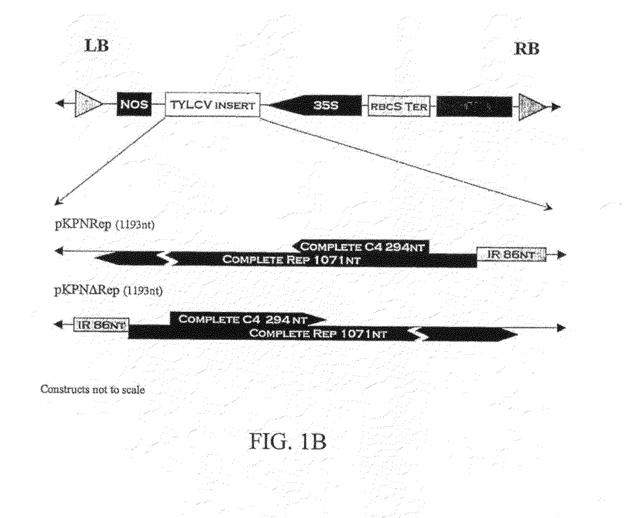

Tomato yellow leaf curl virus (TYLCV) Rep gene (C1) cDNAs corresponding to full length Rep or truncated Rep gene fragments were PCR-amplified from the extracts of TYLCV-infected tomato plants from Florida with various primer pairs containing restriction endonuclease sites for subsequent cloning (Table 1). PCR was conducted under conditions as follows: 93.5° C. for 45 sec, then 35 cycles of 54° C. for 45 sec, 54 to 72° C. ramping for 1 min, 72° C. for 1 min. The reaction mixture was 10×PCR Buffer (500 mM KCl, 100 mM Tris-HCl, pH 9.0, and 1% Triton® X-100), 12.5 mM dNTPs (Promega Corp., Madison, Wis.), 1 mM spermidine trihydrochloride (USB Corp, Cleveland, Ohio), 2.5 mM MgCl₂, and 1 unit Taq DNA polymerase (Promega Corp., Madison, Wis.). PCR products were first cloned into the pGEM-T vector (Promega Corp., Madison, Wis.) for propagation of the target sequence. The amplified and cloned TYLCV Rep sequences were cleaved from the vector and directly ligated into binary vector pKYLX71:35S² (An, 1987; Maiti et al., 1993) (FIG. 1A) or a modified vector with the non-enhanced 35S promoter (pKPN) in either sense or antisense (Δ) orientations (FIG. 1B). In this study, eight expression vectors were constructed; they are designated: pKY2/5Rep, pKYΔ2/5Rep, pKYC4, pKYΔC4, pKYRep, pKYRepΔ2/5Rep, pKPNRep, and pKPNΔRep. To construct pKPNRep and pKPNΔRep, the enhanced 35S promoter of pKYLX was removed by EcoRI and ClaI digestions and replaced by the pBI121 expression cassette (Clontech, Palo Alto, Calif.) containing regular 35S promoter and NOS terminator (FIG. 1B).

### Description of the Constructs.

Following is a description of the constructs used in this study. Diagrams of the constructs showing the details of their composition are shown in FIGS. 1A and 1B.

pKY2/5Rep (also referred to herein as “2/5Rep” construct) (SEQ ID NO: 16): 80 nucleotides of the IR (nucleotides 2696 to 2616 of GenBank accession no. AY530931) plus 426 nucleotides of the 5′ terminus of the TYLCV Rep gene;

pKYΔ2/5Rep (also referred to herein as “Δ2/5Rep” construct) (SEQ ID NO: 17): 595 nucleotides of the 5′ terminus of the TYLCV Rep gene (nucleotides 2021 to 2615 of GenBank Accession No. AY530931) (encompasses the entire C4 gene) in the antisense orientation, followed by 85 nucleotides of the IR (nucleotides 2616 to 2701) in the antisense orientation;

pKYRepΔ2/5Rep (also referred to herein as “RepΔ2/5Rep” construct) (SEQ ID NO: 18): 80 nucleotides of the IR (nucleotides 2696 to 2616 of GenBank accession no. AY530931), followed by the entire Rep gene (nucleotides 2615-1501 of GenBank accession no. AY530931), followed by 34 nucleotides downstream of the 3′ end of the Rep gene, operatively linked to the Δ2/5Rep as above. The IR is included twice in the construct—at the 5′ end (80 nucleotides) and at the 3′ end (85 nucleotides) of the transgene;

pKYRep (also referred to herein as “Rep” construct) (SEQ ID NO: 19): 80 nucleotides of the IR (nucleotides 2696 to 2616 of GenBank accession no. AY530931), followed by the entire Rep gene (nucleotides 2615-1501 of GenBank accession no. AY530931), followed by 34 nucleotides downstream of the 3′ end of the Rep gene (includes the entire C4 gene);

pKNRep (also referred to herein as “NRep” construct) (SEQ ID NO: 20): (non-enhanced 35S promoter=N) 87 nucleotides of the IR, followed by the entire Rep gene, followed by 29 nucleotides 3′ to the Rep gene;

pKNΔRep (also referred to herein as “NΔRep” construct) (SEQ ID NO: 21): (antisense of NRep) 34 nucleotides downstream of the 3′ end of the Rep gene, followed by the entire Rep gene, followed by 107 nucleotides of the all in antisense orientation;

pKYC4 (also referred to herein as “C4” construct) (SEQ ID NO: 22): 80 nucleotides of the IR, followed by 151 nucleotides of the Rep gene, then the entire C4 gene, followed by 151 nucleotides 3′ to the C4 gene; and

pKYΔC4 (also referred to herein as “ΔC4” construct) (SEQ ID NO: 23): (reverse orientation of C4) 151 nucleotides 3′ to the C4 gene, followed by the entire C4 gene, followed by 102 nucleotides 5′ to the C4 gene, all in the antisense orientation. The ΔC4 construct does not include any nucleotides of the IR.

### Tomato Transformation and Screening.

Binary vectors containing the constructs were transformed into Agrobacterium tumefaciens LBA 4404 by a tri-parental mating approach (An, 1987). Cotyledonary tissues of tomato seedlings from Florida breeding lines Fla. 7613 and Fla. 7324 (provided by J. W. Scott) were transformed by A. tumefaciens LBA 4404 harboring the binary vector. Approximately 500-600 explants were used for each construct during transformation experiments. Putative transformants were screened on Murashige-Shoog (MS) media containing kanamycin (100 μg/ml) (Horsch et al., 1985). Regenerated (transgenic) plants (R₀) were grown in the greenhouse for transgene confirmation and seed production.

### Molecular Analysis of Transgenes Via PCR Screening of Transformants.

DNA was extracted from R₀ regenerated plants (Presting et al., 1995) and was used for PCR amplification of the transgene. PCR was conducted under the same conditions as described above and with the same primer pair as used for transgene construction.

A different set of primer pairs was used to identify and distinguish the different constructs in R₁- and R₂-generation transformed plants. These would confirm whether the transgenes were present and allow the detection of different constructs in the same plant should any cross-pollination have occurred. The primer pairs and the expected size of the amplicon obtained with each construct and primer pair are shown in Table 2. Five pairs of primers were developed in which one primer would bind to the non-TYLCV Rep regions of the construct, and the other would bind in the Rep gene sequence. The primers used were: JAP94 (5′-TCTCCATCCATTTCCATTTCAC-3′) (SEQ ID NO: 8), which binds in the Rubisco terminator; JAP28 (5′-GATAGTGGAAAAGGAAGG-3′) (SEQ ID NO: 9), which binds in the CaMV 35S promoter; JAP62 (5′-GGATAAGCACATGGAGATGTGG-3′) (SEQ ID NO: 10) and EH313 (5′-GGATTTACTGCCTGAATTG-3′) (SEQ ID NO: 11), which bind in the middle of the antisense sequence of the TYLCV Rep gene; JAP83 (5′-TCCCACTATCCTTCGCAAGACC-3′) (SEQ ID NO: 12), which binds in the CaMV 35S promoter; JAP84 (5′-ATCATCGCAAGACCGGCAAC-3′) (SEQ ID NO: 13), which binds in the NOS terminator; and EH316 (5′-AAATAGCCATTAGGTGTCC-3′) (SEQ ID NO: 14), and EH337 (5′-CCCAATTGTTCTCTCTCTA-3′) (SEQ ID NO: 15) (Table 1), which bind in the TYLCV Rep gene and were used to obtain some of the inserts.

All primer sets described above used the same PCR program to amplify products. The reaction conditions were as described above for the construction of the transgenes. The PCR program has an initial denaturing step at 92° C. for 2 min, followed by 35 cycles of 1 min at 94° C. for denaturing, 20 sec at 60° C. for annealing, and 30 sec at 72° C. for elongation, ending with an extended elongation at 72° C. for 7 min followed by an indefinite hold at 4° C. (GeneAmp PCR System 9700, PE Applied Biosystems, Foster City, Calif.).

### Plant Inoculation and Virus Detection.

TYLCV and Whitefly Cultures. The TYLCV isolate used in this study was obtained originally from an infected tomato plant growing in a commercial planthouse in Homestead, Fla. in 1997 (Polston et al., 1997). This virus has been fully sequenced (GenBank accession no. AY530931) and has been identified as an isolate of TYLCV first recognized in Israel (GenBank accession no. X15656 (Cohen et al., 1994). Adult populations of B. tabaci biotype B, which were identified by isozyme analysis, were used in this study. Whiteflies were reared on TYLCV-infected tomato (“Florida Lanai”) in a growth chamber (23 to 25° C., 40% relative humidity, 13 h light). Whiteflies of mixed age and gender were obtained from infected plants by gently tapping the plants to dislodge whiteflies. Those that fell or flew onto a yellow plastic card were collected with an aspirator.

Inoculation of TYLCV to R₁ Generation Plants. Approximately 15 R₁ progeny of every R₀ plant were evaluated for resistance to TYLCV in the greenhouse. Tomato seeds were germinated in Todd planter flats (128 cells/flat) and were inoculated at the four- to six-true-leaf stage in a greenhouse at the Gulf Coast Research and Education Center, Bradenton, Fla. Todd planter flats containing the tomato seedlings were placed on shallow trays for watering and enclosed in white nylon organdy bags with an internal support made of PVC plastic pipe. Whiteflies were collected from TYLCV-infected plants. An average of 10 adult whiteflies per test plant were added two times 1 week apart. Tomato seedlings were watered daily by filling the tray with water poured through the sides of the bags so that whitefly feeding was not disturbed, and so that whitefly escape during the inoculation period was minimized. Whiteflies were allowed to remain on test plants for 14 days and then whiteflies were killed by applying a drench of imidacloprid (Admire 2 F at 4 ml per liter of water) to the test plants. The tomato seedlings then were moved to a large enclosed greenhouse with natural light.

Both transformed and non-transformed plants were whitefly-inoculated with TYLCV. Each tray contained six lines (five lines of interest plus the non-transformed control) with 15 plants each. Plants from the different lines were evenly distributed throughout the tray in order to allow a more effective inoculation.

Inoculation of TYLCV to R₂ Generation Plants. R₂-generation plants from 13, 5, and 2 R₁-generation plants transformed with 2/5Rep, Δ2/5Rep, and the RepΔ2/5Rep transgenes, respectively, were evaluated for resistance to TYLCV under field conditions. In the case of the 2/5Rep construct, the progeny of 13 R₁-generation plants selected from a total of 31 R₁-generation parents were evaluated. These 13 were selected to represent lines where there was low, intermediate, and high frequencies of resistance. Plants were seeded as for R₁-generation plants. Plants were inoculated at the four- to six-true-leaf stage with non-transformed plants (20 per flat) located randomly in the flats in order to measure the inoculation rate. Plants were inoculated in small-enclosed greenhouses using whiteflies which were shaken from TYLCV-infected tomato plants each morning for a 3-week inoculation period. Additional infected plants containing whiteflies were added one week into the inoculation period to increase the supply of viruliferous whiteflies. Seedlings were lightly brushed and trays were rotated twice each day to decrease whitefly aggregation. The seedlings were hardened off during the last week of the inoculation period. The inoculation period was ended with an application of imidacloprid. Plants were then planted in the field.

Tomato seedlings were planted in the field at a plant spacing of 0.6 m and a row spacing of 1.5 m. Imidacloprid (0.03% Admire 2 F) and fertilizer (4 g of 20-20-20 per liter) were added in the setting water. Standard production practices were followed for the remainder of the season. A total of 23 R₂-generation lines plus two non-transformed lines (Fla. 7613 and Fla. 7324) were evaluated. Approximately 60 plants per line were evaluated in a randomized complete block design, with approximately 20 plants per block.

### Determining Resistance to TYLCV In R₁- and R₂-Generation Transformed Tomato Plants.

R₁ Generation. Plants were screened for virus symptoms beginning 3 weeks after the end of the inoculation access period and continuing weekly until plants were discarded after seed collection. Tissue samples of young leaves were collected from all plants at 4, 8, and 12 weeks after the inoculation access period and were tested by spot hybridization (4 and 8 weeks) and by PCR (12 weeks). Plants were screened for the presence or absence of TYLCV symptoms. The probe used in the nucleic acid spot hybridization assay was a 351-nucleotide portion of the TYLCV genome that did not overlap with the TYLCV sequences used in the transgenes. The probe DNA is composed of nucleotides 2750 to 320, which represent a region 10 nucleotide upstream of the stemloop to 18 nucleotide into the coat protein gene. Standard hybridization conditions and stringent washing conditions consisted of 65° C. hybridization overnight; 2 washes of 2×SSC (1×SSC is 0.15 M NaCl plus 15 mM sodium citrate) at room temperature (RT) for 5 min each; two washes 0.2×SSC, 1.0% sodium dodecyl sulfate (SDS) at 65° C. for 15 min each; followed by two washes of 0.1×SSC 1.0% SDS at RT for 15 min each. PCR screening used primers PCRv181 and PAL1c496 which would produce an amplicon of approximately 500 nucleotide (Rojas et al., 1993). Plants infected with TYLCV were discarded. Resistant plants were tested for the presence of the transgene at 12 weeks after inoculation and once more just before seed was collected.

R₂-Generation. Resistance was evaluated by symptom expression, dot spot hybridization and PCR. Tissue samples of young leaves were collected from all plants at 4, 8, and 12 weeks after the inoculation and were tested by nucleic acid spot hybridization (4 and 8 weeks) and by PCR (12 weeks). Plants were tested for infection with TYLCV as described for the R₁ generation.

Any element of any embodiment disclosed herein can be combined with any other element or embodiment disclosed herein.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1

### R₀ Generation Plants

Plants transformed with all eight constructs were obtained (FIG. 1) but the number of viable (seed-bearing) transformants for each construct varied (Table 3). Plants transformed with the C4 construct had a highly altered phenotype with greatly reduced lateral growth and flowers growing from the main stem. No viable transformants were obtained with this construct. The R₁-generation progeny of all viable R₀ plants were evaluated with the exception of R₀ plants containing the 2/5Rep transgene. Because of the large number of R₀ 2/5Rep plants, only the progenies of 34 out of 46 lines were evaluated (Table 4).

### Example 2

### Greenhouse Evaluation of R₁ Generation Plants

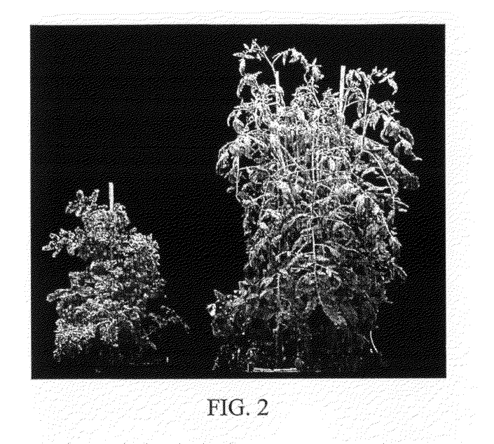

An average of 15 plants in each R₁-generation line were evaluated for resistance to TYLCV (Table 4). The transgene was transferred to the next generation in a majority of the R₁-generation lines, with the exception of the Rep and NΔRep, where only one line of each had transformed plants. The majority of the R₁-generation lines transgenic for either the 2/5Rep (21/31) or the Δ2/5Rep (15/19) had plants with no symptoms of TYLCV (FIG. 2). For the RepΔ2/5Rep transgene, only 3/10 R₁ lines were symptomless. No TYLCV DNA could be detected by either spot hybridization or PCR in symptomless plants. These plants had normal phenotypes. No resistant plants were observed in R₁-generation lines transformed with ΔC4, Rep, NRep, or NΔRep.

The resistance obtained using the 2/5Rep, Δ2/5Rep, and the RepΔ2/5Rep constructs varied in frequency among the lines transformed with the same construct (Table 4). There was a significant amount of segregation for resistance in the R₁-generation lines as expected. The mean frequency of resistant plants among all lines evaluated varied from 26.3% for Δ2/5Rep-transformed plants to 47.7% for RepΔ2/5Rep-transformed plants. In plants that contained the transgene, the frequency of resistance was much higher with a low of 60.7% for Δ2/5Rep-transformed plants and a high of 79.7% for 2/5Rep-transformed plants. Not all transformed plants were resistant to TYLCV. Seed was collected from selected resistant plants for evaluation in subsequent generations.

Some R₁ plants were observed to have a mildly altered horticultural phenotype. Fruit were not correctly shaped, and leaves showed some mild epinasty. These altered phenotypes were not associated with any one transgene and appeared at a low incidence in some lines. These off types were not selected for advancement to the next generation.

### Examples 3

### Field Evaluation of R₂ Generation Plants

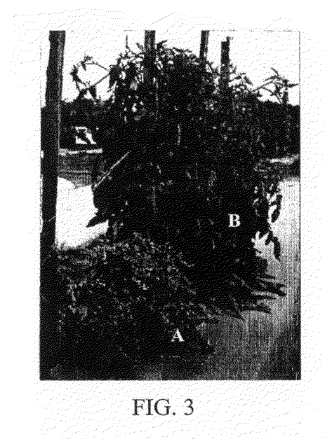

R₂-generation plants from 13, 5, and 2 R₁-generation plants transformed with 2/5Rep, Δ2/5Rep, and the RepΔ2/5Rep transgenes, respectively, were evaluated for resistance to TYLCV under field conditions. Non-transformed plants were 100% infected with TYLCV by 4 weeks after the end of the inoculation access period. R₂-generation plants were considered resistant when they showed no symptoms of TYLCV infection, tested negative for TYLCV DNA by nucleic acid hybridization at 4, 8, and 12 weeks after the inoculation period, and tested negative for TYLCV DNA by PCR at 12 weeks after the inoculation period. Resistant plants (FIG. 3) were detected in all lines evaluated. As expected, plants in this generation were still segregating for resistance. The frequency of resistant plants varied among R₂-generation lines transformed with the same construct (Table 5). The highest frequency of resistance, 80%, was observed in a line transformed with 2/5Rep. However, plants transformed with the RepΔ2/5Rep construct had the highest mean frequency of resistance (69.6%). Lines transformed with the Δ2/5Rep construct had the lowest mean frequency of resistance (22.3%) compared to the other two constructs. Resistant plants were tested for the presence of the transgene by PCR to confirm both its presence and identity. All resistant plants had a transgene and the identity was confirmed. No altered phenotypes (as observed in the R₁-generation in the greenhouse) were observed in the R₂-generation plants in the field.

### Example 4

### TYLCV Resistance

The best resistance to TYLCV infection was obtained using constructs that contained IR and 2/5 Rep gene sequences of TYLCV in either the sense or antisense orientation (Table 1). Resistance was observed at high frequency in both the R₁ and R₂ generations. The absence of detectable viral DNA by either nucleic acid hybridization or PCR in TYLCV-inoculated plants suggested that the resistance may be immunity.

Several other transgenes (ΔC4, Rep, NRep, and NΔRep) tested did not produce TYLCV resistance in plants. However, in some cases, the limited number of transformed plants tested does not preclude possible resistance if more transformants had been obtained. R₀ plants that were transformed successfully with the C4 transgene failed to produce viable seed. The altered phenotype in these plants suggests that the C4 transgene product may have been toxic and caused the plants to be sterile. The presence of the C4 gene sequence in the transgenes containing the entire Rep gene (i.e., NRep and NΔRep) also may have suppressed the transformation efficiency of this construct (Table 3). The difficulty in obtaining plants transformed with the Rep transgene may have other causes. Day et al. (1991) were unable to obtain tobacco plants transformed with a full length Rep of TGMV and Bendahmane and Gronenborn (1997) were unable to regenerate plants that expressed a sense copy of the complete TYLCSV Rep. Bendahmane and Gronenborn (1997) ascribed this failure to possible deleterious affects of the Rep protein on the cell.

Transformants that contained the ΔC4 transgene, which only differed from the Δ2/5Rep construct by the absence of IR sequences and a deletion of 48 nucleotide from the 5’ end of the Rep sequence, did not produce resistance in the R₁ generation. This indicated that sequences of the IR and possibly the 5′ end of the Rep may be correlated with TYLCV immunity. The presence of a non-enhanced promoter (constructs NRep and NΔRep) did not increase the frequency of resistant plants. An antisense Rep construct with an enhanced promoter has been shown by others to generate resistance (Day et al., 1991); thus, the failure of the NΔRep construct to be effective suggests that the resistance may be dose dependent (Tenllado et al., 2001).

Studies were focused on the most basic of the constructs, the 2/5Rep. Progeny of 13 R₁-generation plants selected from a possible 31 R₁-generation parents were evaluated in the field and all showed similar strong resistance or immunity to TYLCV. Under field conditions the plants were exposed to possible natural variants of TYLCV and to possible resistance suppression by infection with other viruses. Plants tested during three growing seasons for TYLCV resistance all showed normal-looking phenotypes and fruit.

The resistance/immunity associated with the Δ2/5Rep transgene suggests that the expression of the Rep protein may not be necessary for resistance. This may be in contrast to the studies of Brunetti et al. (Brunetti et al., 2001; Brunetti et al., 1997) which found a strong correlation between TYLCSV resistance in transgenic tomato and high levels of Rep protein expression. However, the resistance reported by Brunetti et al., 1997, 2001 has other characteristics that distinguish it from 2/5 TYLCV Rep resistance. Their resistance is not stably inherited and the resistance manifests itself as a delay in symptom expression rather than immunity.

The three TYLCV Rep gene constructs that provided TYLCV resistance all contained sequences (IR) upstream of the expected start of the Rep gene transcript. The upstream sequence contains sequence specific elements (iterons) for Rep protein binding during the rolling circular replication of begomoviruses (Gutierrez, 2002). Iterons consist of 8 to 12 nucleotide-direct and -inverted repeats upstream from the transcription start of the Rep (Arguello-Astorga et al., 2001). TYLCV iterons are arranged such that there is one in the sense direction and three in the opposite direction. The inverted repeats are capable of forming hairpin structures when transcribed to RNA. In addition, the RepΔ2/5Rep, a construct which would be expected to form a large hairpin structure in the transcript (the antisense 2/5Rep segment binding to sense segment of the 5′ end of the full length Rep leaving nucleotides 1505 to 2020 to form a large loop), also showed high frequencies of immunity in transgenic lines. The VMYMV interfering DNA constructs described by Pooggin et al. also contain the iteron region of VMYMV (Pooggin et al., 2003). Constructs that express both sense and antisense RNA and thus form double-stranded RNA hairpin loops have been shown to be very effective in gene silencing, often generating immunity (Smith et al. 2000; Wang et al., 2000; Wesley et al., 2001). Both the hairpin structures (Han et al., 2002; Wesley et al., 2001) and direct and indirect repeats (Ma et al., 2002) have been shown to be efficient triggers of post-transcriptional gene silencing (PTGS). Han and Grierson (2002) found that adding inverted repeats to the 5′ end of the untranslated region of their transgene led to stronger silencing.

The virus resistance attributed to PTGS is often expressed as immunity, is associated with transgene transcript suppression, and is characterized as narrow spectrum (Chicas et al., 2001). Characteristics of TYLCV resistance expressed by constructs of the present invention implicate gene silencing. The resistance associated with the 2/5Rep transgene showed immunity and transgene transcript suppression (Freitas-Astua, 2001). The transgenic resistance has characteristics of post transcriptional gene silencing (PTGS)—the transgenic plants show immunity that is sequence specific; transgenic plants show recovery from disease symptoms when infected with distantly related TYLCV strain (TYLCV-Mld, see below); the resistance is transmitted through a graft (see below); the resistance can be overcome when coinfected with an RNA virus (Tomato mosaic virus) known to have a PTGS suppressor; and there is suppression of transgene transcript level in virus resistant transgenic plants (Freitas-Astua, 2001).

### Example 5

### Graft Transmissions of TYLCV Rep-Mediated Resistance

TYLCV resistant R₁ and R₂ generation plants transformed with the 2/5 TYLCV Rep construct showed no virus symptoms and no virus was detected after inoculation with TYLCV. Studies were conducted to determine if transgenic plants were capable of transferring resistance to susceptible plants by grafting. When a 2/5 TYLCV Rep transgenic plant was inoculated and grafted to a non-inoculated susceptible scion, no symptoms or virus DNA was detected 12 wks after the graft was established. When a 2/5 TYLCV Rep transgenic plant was inoculated and grafted to an inoculated susceptible scion, the symptoms became less with time as the scion “recovered” and was cleared of the virus. When a 2/5 TYLCV Rep transgenic plant was grafted to an inoculated susceptible scion, the symptoms of the scion were reduced over time. These results support the conclusion that resistance obtained using the 2/5 Rep construct is an induced translocatable response able to interfere with an established infection and reduce symptom expression in non-transgenic tissue.

### Example 6

### TYLCV Rep Derived Resistance to Two Strains of TYLCV

Plants transformed with the 2/5Rep, Δ2/5Rep or with RepΔ2/5Rep constructs were tested for resistance to Tomato mottle virus (ToMoV) in Florida, and to a mixed infection of TYLCV and TYLCV-Mld in Israel by M. Lapidot by whitefly inoculation in the greenhouse. All constructs produced resistance to TYLCV similar to the resistance to TYLCV-[Fl] (Table 2). No symptoms were produced and viral genomic DNA was not detected by PCR. Transgenic lines that were resistant to TYLCV-[Fl] also showed resistance to TYLCV-Mld. The TYLCV 2/5Rep lines tested showed a recovery response to TYLCV-Mld. Plants transformed with RepΔ2/5Rep constructs were highly resistant to TYLCV-[Fl], TYLCV and TYLCV-Mid. This breadth of resistance to TYLCV-Mld strain was unexpected since there is only 76% sequence identity between the 2/5 TYLCV Rep construct and TYLCV-Mld. None of the lines were resistant to unrelated (sequence) ToMoV.

The resistance to TYLCV demonstrated herein is superior to that of commercially available resistances derived from wild Lycopersicon species due to the absence of virus in resistant plants, single dominant gene inheritance, and the ability of the plants to resist infection in spite of early and intense inoculation pressure. The disclosure herein differs from other transgenic Geminivirus resistance reports involving the Rep gene in that viruliferous whiteflies were used for challenge inoculation instead of agroinoculation or biolistic inoculation, and in that plants were screened for TYLCV resistance under field conditions, and in the use of the IR sequences in the subject transgene constructs. This work is distinguished from other reports of TYLCV and TYLCSV Rep derived resistance in that the resistance derived from 2/5 TYLCV Rep gene is stably inherited and the resistance is very strong—after inoculation no TYLCV is detected at the site of inoculation or at any other site on the plant. Another distinctive feature of the present invention is the evaluation of the transgenic resistance for TYLCV under field conditions. The minimal-sized, 2/5 Rep constructs generated resistance to TYLCV that is more robust than commercially available resistances. This construct was also tested in transgenic tobacco and found to confer similar immunity to TYLCV, indicating that this construct is a strong inducer of TYLCV resistance in independent transformation events and in a different genetic background (Freitas-Astua, 2001). The presence of the transgenes was not associated with an altered horticultural phenotype. Further development of this immunity has great potential in the commercialization of cultivars with superior resistance to whitefly-transmitted geminiviruses (begomoviruses).

It should be understood that the examples and embodiments described herein are for illustrative purposes only and that various modifications or changes in light thereof will be suggested to persons skilled in the art and are to be included within the spirit and purview of this application.

TABLE 1

Expression vectors and PCR primers used to construct the
transformation cassettes.

| | | | | |
|---|---|---|---|---|
| | | | | Length |
| | | | | of |
| | | Primer | Restrict- | insert |
| Construct | Primer Sequence¹ | Id. No. | ion Sites | (bp) |

| | | | | |
|---|---|---|---|---|
| pKY2/5Rep | 5′-CCAAGCTTAGCCATTAGGTGTCCAAG-3′ | EH 312 | Hind III | 506 |
| | (SEQ ID NO: 1) | | | |
| | 5′-CTCTCGAGGGATTTACTGCCTGAATTG-3′ | EH 313 | Xho I | |
| | (SEQ ID NO: 2) | | | |
| pKYΔ2/5Rep | 5′-CTCTCGAGCGACCCACTCTTCAAGTTC-3′ | EH 315 | Xho I | 681 |
| | (SEQ ID NO: 3) | | | |
| | 5′-GCTCTAGAAAATAGCCATTAGCTGTCC-3′ | EH 316 | Xho I | |
| | (SEQ ID NO: 4) | | | |
| pKYC4 | 5′-CTCTCGAGCGACCCACTCTTCAAGTTC-3′ | EH 315 | Xho I | 676 |
| | (SEQ ID NO: 3) | | | |
| | 5′-CCAAGCTTAGCCATTAGGTGTCCAAG-3′ | EH 312 | Hind III | |
| | (SEQ ID NO: 1) | | | |
| pKYΔC4 | 5′-CTCTCGAGCGACCCACTCTTCAAGTTC-3′ | EH 315 | Xho I | 547 |
| | (SEQ ID NO: 3) | | | |
| | 5′-CCTCTAGACCCAATTGTTCTCTCTCTA-3′ | EH 337 | Xba I | |
| | (SEQ ID NO: 5) | | | |
| pKYRep | 5′-CCAAGCTTAGCCATTAGGTGTCCAAG-3′ | EH 312 | Hind III | 1188 |
| | (SEQ ID NO: 1) | | | |
| | 5′-CTCTCGAGGTAGTATGAGCAGCCACAG-3′ | EH 330 | Xho I | |
| | (SEQ ID NO: 6) | | | |
| pKYRepΔ2/5Rep | 5′-CCAAGCTTAGCCATTAGGTGTCCAAG-3′ | EH 312 | HindIII | 1871 |
| | (SEQ ID NO: 1) | | | |
| | 5′-CTCTCGAGGTAGTATGAGCAGCCACAG-3′ | EH 330 | Xho I | |
| | (SEQ ID NO: 6) | | | |
| | 5′-CTCTCGAGGGACCCACTCTTCAAGTTC-3′ | EH 315 | Xho I | |
| | (SEQ ID NO: 3) | | | |
| | 5′-GCTCTAGAAAATAGCCATTAGCTGTCC-3′ | EH 316 | Xba I | |
| | (SEQ ID NO: 4) | | | |
| pKPNRep | 5′-GTGAGCTCTAGTATGAGCAGCCACAG-3′ | EH 317 | Sst I | 1190 |
| | (SEQ ID NO: 7) | | | |
| | 5′-GCTCTAGAAAATAGCCATTAGGTGTCC-3′ | EH 316 | Xba I | |
| | (SEQ ID NO: 4) | | | |
| pKPNΔRep | 5′-GCTCTAGAAAATAGCCATTAGGTGTCC-3′ | EH 316 | Xba I | 1193 |
| | (SEQ ID NO: 4) | | | |
| | 5′-GTGAGCTCTAGTATGAGCAGCCACAG-3′ | EH 317 | Sst I | |
| | (SEQ ID NO: 7) | | | |

¹Underlined sequences represent restriction sites.

TABLE 2
Primers used to detect transgenes in R₁- and R₂-generation transformed
plants and expected sizes generated by each transgene^{a}.

| | JAP94, | JAP28, | JAP94, | JAP83, | JAP28, |
|---|---|---|---|---|---|
| Construct | EH337 | EH316 | EH313 | JAP84 | JAP62 |
| pKY2/5Rep | 592 | — | — | — | 539 |
| pKYΔ2/5Rep | — | 971 | 698 | — | — |
| pKYC4 | 761 | — | — | — | 539 |
| pKYΔC4 | — | — | 564 | — | — |
| pKYRep | 1275 | — | — | — | 539 |
| pKYRepΔ2/5Rep | — | — | 698 | — | 539 |
| pKPNRep | — | — | — | 1343 | 539 |
| pKPNΔRep | — | — | — | 1343 | — |

^{a}One primer binds in the non-TYLCV Rep regions of the construct, the other binds in the Rep. JAP94 binds in the Rubisco terminator, JAP28 binds in the CaMV 35S promoter, JAP83 binds in the CaMV 35S promoter, JAP84 binds in the NOS terminator, EH313 and JAP62 bind in the antisense sequence of the Rep, and EH316 and EH337 bind in the TYLCV Rep gene;
— means no amplicon produced.

TABLE 3
Number of R₀-generation tomato plants
transformed with TYLCV Rep gene constructs from
which seed was obtained.

| Transgene^{a} | R₀ Plants^{b} |
|---|---|
| 2/5Rep | 46 |
| Δ2/5Rep | 24 |
| RepΔ2/5Rep | 17 |
| ΔC4 | 18 |
| C4 | 0 |
| Rep | 1 |
| NRep | 10 |
| NΔRep | 7 |

^{a}Approximately 500 to 600 explants were used for each construct during transformation attempts.
^{b}Number of plants that produced seed.

TABLE 4
Greenhouse evaluation of R₁-generation tomato plant lines transformed with
TYLCV gene constructs for resistance to infection by TYLCV.

| | Number of R₁-generation | | | | Resistance | | |
|---|---|---|---|---|---|---|---|
| | | | | Lines | | | Mean |
| | | | | With | | | Frequency |
| | | | Lines | Transgene | | Mean | in Plants with |
| | Plants | Lines | with | And | Frequency | Frequency | Transgene |
| Transgene | Evaluated^{a} | Tested | Transgene | Resistance | (%) | (%) | (%) |
| 2/5Rep | 437 | 34 | 31 | 21 | 7-91 | 42.7 | 79.7 |
| Δ2/5Rep | 258 | 23 | 19 | 15 | 8-50 | 26.3 | 60.2 |
| RepΔ2/5Rep | 166 | 14 | 10 | 3 | 14-69 | 47.7 | 77.7 |
| ΔC4 | 192 | 18 | 14 | 0 | 0 | 0 | 0 |
| Rep | 28 | 2 | 1 | 0 | 0 | 0 | 0 |
| NRep | 152 | 12 | 8 | 0 | 0 | 0 | 0 |
| NΔRep | 40 | 3 | 1 | 0 | 0 | 0 | 0 |

^{a}Excludes plants that died during the evaluation.

TABLE 5
Field evaluation of R₂-generation tomato plants transformed with
different TYLCV gene constructs for resistance
to infection by TYLCV.

| | Number of R₂-generation | | | Resistance | |
|---|---|---|---|---|---|
| | Lines | Plants | Lines with | Frequency | Mean |
| Transgene | Tested | Tested¹ | Resistance | (%) | Frequency (%) |
| 2/5Rep | 13 | 347 | 13 | 26.6-80.0 | 53.8 |
| Δ2/5Rep | 4 | 135 | 5 | 12.5-50.0 | 27.8 |
| RepΔ2/5Rep | 2 | 50 | 2 | 66.9-72.2 | 69.6 |

^{a}Excludes plants that died during the evaluation.

TABLE 6

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

TABLE 7

| Letter Symbol | Amino Acid |
|---|---|
| A | Alanine |
| B | Asparagine or |
| | aspartic acid |
| C | Cysteine |
| D | Aspartic Acid |
| E | Glutamic Acid |
| F | Phenylalanine |
| G | Glycine |
| H | Histidine |
| I | Isoleucine |
| K | Lysine |
| L | Leucine |
| M | Methionine |
| N | Asparagine |
| P | Proline |
| Q | Glutamine |
| R | Arginine |
| S | Serine |
| T | Threonine |
| V | Valine |
| W | Tryptophan |
| Y | Tyrosine |
| Z | Glutamine or |
| | glutamic acid |

### REFERENCES

U.S. Pat. No. 5,106,739
U.S. Pat. No. 5,625,136
U.S. Pat. No. 5,034,322
U.S. Pat. No. 6,455,760
U.S. Pat. No. 6,696,623
U.S. Published Patent Application No. 20040078841
U.S. Published Patent Application No. 20040067506
U.S. Published Patent Application No. 20040019934
U.S. Published Patent Application No. 20030177536
U.S. Published Patent Application No. 20030084486
U.S. Published Patent Application No. 20040123349
An, G. (1987) “Binary Ti vectors for plant transformation and promoter analysis” Methods Enzymol. 153:292-305.
Arguello-Astorga, G. R., and Ruiz-Mendoza, R. (2001) “An iteron-related domain is associated to Motif 1 in the replication proteins of geminiviruses: identification of potential interacting amino acid-base pairs by a comparative approach” Arch. Virol. 146:1465-1485.
Altschul et al. (1990) J. Mol. Biol. 215:402-410.
Altschul et al. (1997) Nucl. Acids Res. 25:3389-3402.
Beltz, G. A., K. A. Jacobs, T. H. Eickbush, P. T. Cherbas, and F. C. Kafatos (1983) Methods of Enzymology, R. Wu, L. Grossman and K. Moldave [eds.] Academic Press, New York 100:266-285.
Bendahmane, M., and Gronenborn, B. (1997) “Engineering resistance against tomato yellow leaf curl virus (TYLCV) using antisense RNA” Plant Molec. Biol. 33:351-357.
Brunetti, A., Tavazza, R., Noris, E., Lucioli, A., Accotto, G. P., and Tavazza, M. (2001) “Transgenically expressed T-Rep of tomato yellow leaf curl sardinia virus acts as a trans-dominant-negative mutant, inhibiting viral transcription and replication” J. Virol. 75:10573-10581.
Brunetti, A., Tavazza, M., Noris, E., Tavazza, R., Caciagli, P., Ancora, G., Crespi, S., and Accotto, G. P. (1997) “High expression of truncated viral rep protein confers resistance to tomato yellow leaf curl virus in transgenic tomato plants” Molec. Plant-Microbe Interact. 10:571-579.
Carrer, H., P. Maliga (1995) “Targeted insertion of foreign genes into the tobacco plastid genium without physical linkage to the selectable marker” Biotechnology 13:791-794.
Chicas, A., and Macino, G. (2001) “Characteristics of post-transcriptional gene silencing” EMBO Rep. 21:992-996.
Clancy, M. and Hannah, L. C. (2002) “Splicing of the maize Sh1 first intron is essential for enhancement of gene expression, and a T-rich motif increases expression without affecting splicing” Plant Physiol. 130(2):918-29.
Cohen, S., and Nitzany, F. E. (1966) “Transmission and host range of the tomato yellow leaf curl virus” Phytopathology 56:1127-1131.
Cohen, S., and Antignus, Y. (1994) “Tomato yellow leaf curl virus, a whitefly-borne geminivirus of tomatoes” In Advances in Disease Vector Research, New York: Springer-Verlag.
Day, A. G., Bejarano, E. R., Buck, K. W., Burrell, M., and Lichtenstein, C. P. (1991) “Expression of an Antisense Viral Gene in Transgenic Tobacco Confers Resistance to the DNA Virus Tomato Golden Mosaic Virus” Pro. Natl. Acad. Sci. USA 88:6721-6725.
Freitas-Astua, J. (2001) “Characterization of resistance in transgenic tobacco plants expressing begomovirus genes”, Ph. D. Dissertation. University of Florida, Gainesville, Fla.
Friedmann, M., Lapidot, M., Cohen, S., and Pilowsky, M. (1998) “A novel source of resistance to tomato yellow leaf curl virus exhibiting a symptomless reaction to viral infection” J. Amer. Soc. Hort. Sci. 123:1004-1007.
Gilreath, P., Shuler, K., Polston, J. E., Sherwood, T. A., McAvoy, G., Stansly, P. A., and Waldo, E. (2001) “Tomato yellow leaf curl virus resistant tomato variety trials” Proc. Fla. State Hort. Soc. 114:190-193.
Good, X. et al. (1994) “Reduced ethylene synthesis by transgenic tomatoes expressing S-adenosylmethionine hydrolase” Plant Molec. Biol. 26:781-790.
Gutierrez, C. (2002) “Strategies for geminivirus DNA replication and cell cycle interference” Physiol. Molec. Plant Pathol. 60:219-230.
Han, Y., and Grierson, D. (2002) “The influence of inverted repeats on the production of small antisense RNAs involved in gene silencing” Mol. Genet. Genomics 267:629-635.
Horsch, R. B., Fry, J. E., Hoffman, N. L., Wallroth, W., Einholtz, D., Rogers, S. G., and Fraley, R. T. (1985) “A simple and general method for transferring gene into plants” Science 237:1229-1231.
Karlin and Altschul (1990) Proc. Natl. Acad. Sci. USA 87:2264-2268.
Karlin and Altschul (1993) Proc. Natl. Acad. Sci. USA 90:5873-5877.
Kelemen, Z. et al. (2002) “Transformation Vector Based on Promoter and Intron Sequences of a Replacement Histone H3 Gene. A Tool for High, Constitutive Gene Expression in Plants” Transgenic Research 11:69-72.
Lapidot, M., and Friedman, M. (2002) “Breeding for resistance to whitefly-transmitted geminiviruses” Ann. Appl. Biol. 140:109-127.
Lapidot, M., Friedmann, M., Pilowsky, M., Ben-Joseph, R., and Cohen, S. (2001) “The effect of host resistance on Tomato yellow leaf curl virus (TYLCV) on virus acquisition and transmission by its whitefly vector” Phytopathology 91:1209-1213.
Lapidot, M., Friedmann, M., Lachman, O., Yehezkel, A., Nahon, S., Cohen, S., and Pilowsky, M. (1997) “Comparison of resistance level to Tomato yellow leaf curl virus among commercial cultivators and breeding lines” Plant Dis. 81:1425-1428.
Lewin, B. (1985) Genes II, John Wiley & Sons, Inc., p. 96.
Ma, C., and Mitra, A. (2002) “Intrinsic direct repeats generate consistent post-transcriptional gene silencing in tobacco” Plant J. 31:37-49.
Maiti, a. B., Murphy, J. F., Shaw, J. G., and Hunt, A. G. (1993) “Plants that express a potyvirus proteinase gene are resistant to virus infection” Plant Biology 90:6110-6114.
Maniatis, T., E. F. Fritsch, J. Sambrook (1982) Molecular Cloning: A Laboratory Manual, Cold Spring Harbor Laboratory, Cold Spring Harbor, N.Y.
Noris, E., Accotto, G., Tavazza, R., Brunetti, A., Crespi, S., and Tavazza, M. (1996) “Resistance to tomato yellow leaf curl geminivirus in Nicotiana benthaminana plants transformed with a truncated viral C1 gene” Virology 224:130-138.
Pilowsky, M., and Cohen, S. (1990) “Tolerance to tomato yellow leaf curl virus derived from Lycopersicion peruvianum” Plant Dis. 74:248-250.
Polston, J. E., and E. Hiebert. (2001) “Engineered resistance to tomato geminiviruses” pp. 19-22 In: Proc. of the Florida Tomato Institute, September 5 Naples, Fla., P. Gilreath, ed., PRO-518, Univ. of Florida, Gainesville.
Polston, J. E., McGovern, R. J., and Brown, L. G. (1997) “Introduction of tomato yellow leaf curl virus in Florida and implications for the spread of this and other geminiviruses of tomato” Plant Dis. 83:984-988.
Pooggin, M., Shivaprasad, P. V., Veluthambi, K., and Hohn, T. (2003) “RNA targeting of DNA in plants” Nature Biotech. 21:131-132.
Presting, G. C., Smith, O. P., and Brown, C. R. (1995) “Resistance to potato leafroll virus in potato plants transformed with the coat protein gene or with vector control constructs” Phytopathology 85:436-442.
Rojas, M. R., Gilbertson, R. L., Russell, D. R., and Maxwell, D. P. (1993) “Use of degenerate primers in the polymerase chain reaction to detect whitefly-transmitted geminiviruses” Plant Dis. 77:340-347.
Scott, J. W., Stevens, M. R., Barten, J. H. M., Thome, C. H., Polston, J. E., and Schuster, D. J. (1995) “Introgression of resistance to whitefly geminiviruses from Lycopersicon chilense to tomato” In Bemisia: Taxonomy, Biology, Damage, Control, and Management, edited by D. Gerling and R. T. Mayer. Andover, U. K.: Intercept Press.
Smith, N. A., Singh, S. P., Wang, M.-B., Stoutjesdijk, P. A., Green, A. G., and Waterhouse, P. M. (2000) “Total silencing by intron-spliced hairpin RNAs” Nature 407:319-320.
Tenllado, F., and Diaz-Ruiz, J. R. (2001) “Double-stranded RNA-mediated interference with plant virus infection” J. Virol. 75:12288-12297.
Wang, M.-B., Abbot, D. C., and Waterhouse, P. M. (2000) “A single copy of a virus-derived transgene encoding hairpin RNA gives immunity to barley yellow dwarf virus” Molec. Plant Pathol. 1:347-356.
Wesley, S. V., Helliwell, C. A., Smith, N. A., Wang, M., Rouse, D. T., Liu, Q., Gooding, P. S., Singh, S. P., Abbott, D., Stoutjesdijk, P. A., Robinson, S. P., Gleave, A. P., Green, A. G., and Waterhouse, P. M. (2001) “Construct design for efficient, effect and high throughput gene silencing in plants” Plant J. 27:581-590.
Xu, D., McElroy, D., Thornburg, R. W., Wu, R. et al. (1993) “Systemic induction of a potato pin2 promoter by wounding, methyl jasmonate, and abscisic acid in transgenic rice plants” Plant Molecular Biology 22:573-588.
Yang, T. T. et al. (1996) “Optimized Codon Usage and Chromophore Mutations Provide Enhanced Sensitivity with the Green Fluorescent Protein” Nucleic Acid Research 24(22):4592-4593.
Zamir, A. D., Ekstein-Michelson, B. I., Zakay, C. Y., Navot, D. N., Zeidan, E. M., Sarfatti, F. M., Eshed, G. Y., Harel, H. E., Pleban, I. T., Van-Oss, J. H., Kedar, K. N., Rabinowitch, L. H. D., and Czosnek, M. H. (1994) “Mapping and introgression of a tomato yellow leaf curl virus tolerance gene, TY-1” Theor. Appl. Genet. 88:141-146.
Zuo, J. et al. (2002) “Marker-free Transformation: Increasing Transformation Frequency by the Use of Regeneration-Promoting Genes” Current Opinion in Biotechnology 13:173-180.

1. A method for providing resistance to infection by a geminivirus in a plant or plant tissue, said method comprising transforming said plant or plant tissue with a polynucleotide comprising a first polynucleotide that comprises all or a portion of a TYLCV (Tomato yellow leaf curl virus) intergenic region, operatively linked to all or a portion of a TYLCV Rep gene, wherein said first polynucleotide is operatively linked to a second polynucleotide comprising all or a portion of a TYLCV Rep gene in antisense orientation operatively linked to all or a portion of a TYLCV intergenic region in antisense orientation.

2. The method according to claim 1, wherein said TYLCV Rep gene is derived from TYLCV-[Israel] or TYLCV-[Florida].

3-4. (canceled)

5. The method according to claim 1, wherein said first polynucleotide comprises about 50 to 100 nucleotides of a TYLCV intergenic region, operatively linked to a complete TYLCV Rep gene sequence or a fragment thereof, and said second polynucleotide comprises a complete TYLCV Rep gene or a fragment thereof in antisense orientation operatively linked to about 50 to 100 nucleotides of a TYLCV intergenic region in antisense orientation.

6. The method according to claim 1, wherein said first or second polynucleotide comprises about 50 to 100, or about 70 to 90, or about 80 to 85 nucleotides of a TYLCV intergenic region.

7. The method according to claim 1, wherein said first or second polynucleotide comprises about 300 to 700 nucleotides or about 500 to 600 nucleotides of the 5′ terminus of a TYLCV Rep gene.

8. The method according to claim 1, wherein said first polynucleotide comprises from about 50 to 100, or about 70 to 90, or about 80 to 85 nucleotides of a TYLCV intergenic region and from about 300 to 700 nucleotides of the 5′ terminus of a TYLCV Rep gene.

9. The method according to claim 1, wherein said second polynucleotide comprises, in antisense orientation, about 300 to 700 or about 500 to 600 nucleotides of the 5′ terminus of a TYLCV Rep gene and, in antisense orientation, about 50 to 100 or about 80 to 90 nucleotides of a TYLCV intergenic region.

10-16. (canceled)

17. The method according to claim 1, wherein said plant or plant tissue is tomato, tobacco, statice, petunia, lisianthus, tomatillo, pepper, or bean.

18-28. (canceled)

29. The method according to claim 1, wherein said TYLCV Rep gene is derived from a TYLCV strain selected from the group consisting of TYLCV-Mld, TYLCV-IR, and TYLCV-SD.

30. The method according to claim 1, wherein said TYLCV Rep gene is derived from a TYLCV isolate selected from the group consisting of TYLCV-[Alm], TYLCV-[Aic], TYLCV-[CU], TYLCV-[DO], TYLCV-[Fl], TYLCV-[PT], TYLCV-[SA], TYLCV-[Shi], and TYLCV-[ES 7297].

31. A polynucleotide comprising a first polynucleotide that comprises all or a portion of a TYLCV intergenic region, operatively linked to all or a portion of a TYLCV Rep gene, wherein said first polynucleotide is operatively linked to a second polynucleotide comprising all or a portion of a TYLCV Rep gene in antisense orientation operatively linked to all or a portion of a TYLCV intergenic region in antisense orientation.

32. The polynucleotide according to claim 31, wherein said TYLCV Rep gene is derived from a TYLCV strain selected from the group consisting of TYLCV-Mld, TYLCV-IR, and/or TYLCV-SD.

33. The polynucleotide according to claim 31, wherein the said TYLCV Rep gene is an isolate selected from the group consisting of TYLCV-[Alm], TYLCV-[Aic], TYLCV-[CU], TYLCV-[DO], TYLCV-[Fl], TYLCV-[PT], TYLCV-[SA], TYLCV-[Shi], and/or TYLCV-[ES7297].

34. The polynucleotide according to claim 31, wherein said TYLCV Rep gene is derived from TYLCV-[Israel] or TYLCV-[Florida].

35-36. (canceled)

37. The polynucleotide according to claim 31, wherein said first polynucleotide comprises about 50 to 100 nucleotides of a TYLCV intergenic region, operatively linked to a complete TYLCV Rep gene sequence or a fragment thereof, and said second polynucleotide comprises a complete TYLCV Rep gene or a fragment thereof in antisense orientation operatively linked to about 50 to 100 nucleotides of a TYLCV intergenic region in antisense orientation.

38. The polynucleotide according to claim 31, wherein said first or second polynucleotide comprises about 50 to 100, or about 70 to 90, or about 80 to 85 nucleotides of a TYLCV intergenic region.

39. The polynucleotide according to claim 31, wherein said first or second polynucleotide comprises about 300 to 700 nucleotides or about 500 to 600 nucleotides of the 5′ terminus of a TYLCV Rep gene.

40. The polynucleotide according to claim 31, wherein said first polynucleotide comprises from about 50 to 100, about 70 to 90, or about 80 to 85 nucleotides of a TYLCV intergenic region and from about 300 to 700 nucleotides of the 5′ terminus of a TYLCV Rep gene.

41. The polynucleotide according to claim 31, wherein said second polynucleotide comprises, in antisense orientation, about 300 to 700 or about 500 to 600 nucleotides of the 5′ terminus of a TYLCV Rep gene and, in antisense orientation, about 50 to 100 or about 80 to 90 nucleotides of a TYLCV intergenic region.

42-44. (canceled)

45. An expression construct or a vector comprising a polynucleotide comprising a first polynucleotide that comprises all or a portion of a TYLCV intergenic region, operatively linked to all or a portion of a TYLCV Rep gene, wherein said first polynucleotide is operatively linked to a second polynucleotide comprising all or a portion of a TYLCV Rep gene in antisense orientation operatively linked to all or a portion of a TYLCV intergenic region in antisense orientation.

46. (canceled)

47. A cell comprising:
i) a polynucleotide comprising a first polynucleotide that comprises all or a portion of a TYLCV intergenic region, operatively linked to all or a portion of a TYLCV Rep gene, wherein said first polynucleotide is operatively linked to a second polynucleotide comprising all or a portion of a TYLCV Rep gene in antisense orientation operatively linked to all or a portion of a TYLCV intergenic region in antisense orientation; or
ii) an expression construct comprising a polynucleotide comprising a first polynucleotide that comprises all or a portion of a TYLCV intergenic region, operatively linked to all or a portion of a TYLCV Rep gene, wherein said first polynucleotide is operatively linked to a second polynucleotide comprising all or a portion of a TYLCV Rep gene in antisense orientation operatively linked to all or a portion of a TYLCV intergenic region in antisense orientation; or
iii) a vector comprising a polynucleotide comprising a first polynucleotide that comprises all or a portion of a TYLCV intergenic region, operatively linked to all or a portion of a TYLCV Rep gene, wherein said first polynucleotide is operatively linked to a second polynucleotide comprising all or a portion of a TYLCV Rep gene in anti sense orientation operatively linked to all or a portion of a TYLCV intergenic region in antisense orientation.

48. (canceled)

49. A plant seed comprising a plant-cell according to claim 47.

50. A transgenic or transformed plant or plant tissue, or the progeny of said plant or plant tissue, having increased resistance to infection by geminivirus, wherein said plant or plant tissue comprises:
i) a polynucleotide comprising a first polynucleotide that comprises all or a portion of a TYLCV intergenic region, operatively linked to all or a portion of a TYLCV Rep gene, wherein said first polynucleotide is operatively linked to a second polynucleotide comprising all or a portion of a TYLCV Rep gene in antisense orientation operatively linked to all or a portion of a TYLCV intergenic region in antisense orientation; or
ii) an expression construct comprising a polynucleotide comprising a first polynucleotide that comprises all or a portion of a TYLCV intergenic region, operatively linked to all or a portion of a TYLCV Rep gene, wherein said first polynucleotide is operatively linked to a second polynucleotide comprising all or a portion of a TYLCV Rep gene in antisense orientation operatively linked to all or a portion of a TYLCV intergenic region in antisense orientation; or
iii) a vector comprising a polynucleotide comprising a first polynucleotide that comprises all or a portion of a TYLCV intergenic region, operatively linked to all or a portion of a TYLCV Rep gene, wherein said first polynucleotide is operatively linked to a second polynucleotide comprising all or a portion of a TYLCV Rep gene in antisense orientation operatively linked to all or a portion of a TYLCV intergenic region in antisense orientation.

51. The plant or plant tissue according to claim 50, wherein said plant or plant tissue is tomato, tobacco, statice, petunia, lisianthus, tomatillo, pepper, or bean.

52. (canceled)


SEQUENCE LISTING (23 sequences)

SEQ ID NO: 1 - DNA, 26 bp, tomato yellow leaf curl virus
ccaagcttagccattaggtgtccaag

SEQ ID NO: 2 - DNA, 27 bp, tomato yellow leaf curl virus
ctctcgagggatttactgcctgaattg

SEQ ID NO: 3 - DNA, 27 bp, tomato yellow leaf curl virus
ctctcgagcgacccactcttcaagttc

SEQ ID NO: 4 - DNA, 27 bp, tomato yellow leaf curl virus
gctctagaaaatagccattagctgtcc

SEQ ID NO: 5 - DNA, 27 bp, tomato yellow leaf curl virus
cctctagacccaattgttctctctcta

SEQ ID NO: 6 - DNA, 27 bp, tomato yellow leaf curl virus
ctctcgaggtagtatgagcagccacag

SEQ ID NO: 7 - DNA, 26 bp, tomato yellow leaf curl virus
gtgagctctagtatgagcagccacag

SEQ ID NO: 8 - DNA, 22 bp, Unknown
  derived from Rubisco terminator
tctccatccatttccatttcac

SEQ ID NO: 9 - DNA, 18 bp, Cauliflower mosaic virus
gatagtggaaaaggaagg

SEQ ID NO: 10 - DNA, 22 bp, tomato yellow leaf curl virus
ggataagcacatggagatgtgg

SEQ ID NO: 11 - DNA, 19 bp, tomato yellow leaf curl virus
ggatttactgcctgaattg

SEQ ID NO: 12 - DNA, 22 bp, Cauliflower mosaic virus
tcccactatccttcgcaagacc

SEQ ID NO: 13 - DNA, 20 bp, Unknown
  derived from NOS terminator
atcatcgcaagaccggcaac

SEQ ID NO: 14 - DNA, 19 bp, tomato yellow leaf curl virus
aaatagccattaggtgtcc

SEQ ID NO: 15 - DNA, 19 bp, tomato yellow leaf curl virus
cccaattgttctctctcta

SEQ ID NO: 16 - DNA, 506 bp, tomato yellow leaf curl virus
gccattaggtgtcgaggtattagtaagacaccgatacaccgattgccatagagctttgagggacaccgattcatttcaacatgcctcgtttatttaaaatatatgccaagaattatttcctaacatatcccaattgttctctctctaaagaggaagcactttcccaattaaaaaacatagaaaccccaacaaataaaaaatacatcaaagtttgcagagaattccacgagaatggggaaccacatctccatgtgcttatccaattcgaaggcaaataccaatgtaagaaccaacggttcttcgacctggtatccccaaacaggtcagcacatttccatccaaacattcaggcagctaagagctcaacagatgtcaagacctacgtggagaaagacggagacttcattgattttggagttttccaaatcgatggcagatcagctagaggaggtcagcaatctgccaacgacgcatacgccggagcactcaattcaggcagtaaatcc

SEQ ID NO: 17 - DNA, 681 bp, tomato yellow leaf curl virus
gcgacccactcttcaagttcatctggaacttgattaaaagaagaagaaagaaatggagaaacataaacttctaaaggaggactaaaaatcctatctaaatttgaatttaaattatgaaattgtaaaatatagtcctttggggccttttcttttaatatattgagggcctcggatttactgcctgaattgagtgctccggcgtatgcgtcgttggcagattgctgacctcctctagctgatctgccatcgatttggaaaactccaaaatcaatgaagtctccgtctttctccacgtaggtcttgacatctgttgagctcttagctgcctgaatgtttggatggaaatgtgctgacctgtttggggataccaggtcgaagaaccgttggttcttacattggtatttgccttcgaattggataagcacatggagatgtggttccccattctcgtggaattctctgcaaactttgatgtattttttatttgttggggtttctatgttttttaattgggaaagtgcttcctctttagagagagaacaattgggatatgttaggaaataattcttggcatatattttaaataaacgaggcatgttgaaatgaatcggtgtccctcaaagctctatggcaatcggtgtatcggtgtcttactaatacctcgacacctaatggcaattt

SEQ ID NO: 18 - DNA, 1871 bp, tomato yellow leaf curl virus
gccattaggtgtcgaggtattagtaagacaccgatacaccgattgccatagagctttgagggacaccgattcatttcaacatgcctcgtttatttaaaatatatgccaagaattatttcctaacatatcccaattgttctctctctaaagaggaagcactttcccaattaaaaaacatagaaaccccaacaaataaaaaatacatcaaagtttgcagagaattccacgagaatggggaaccacatctccatgtgcttatccaattcgaaggcaaataccaatgtaagaaccaacggttcttcgacctggtatccccaaacaggtcagcacatttccatccaaacattcaggcagctaagagctcaacagatgtcaagacctacgtggagaaagacggagacttcattgattttggagttttccaaatcgatggcagatcagctagaggaggtcagcaatctgccaacgacgcatacgccggagcactcaattcaggcagtaaatccgaggccctcaatatattaaaagaaaaggccccaaaggactatattttacaatttcataatttaaattcaaatttagataggatttttagtcctcctttagaagtttatgtttctccatttctttcttcttcttttaatcaagttccagatgaacttgaagagtgggtcgccgagaacgtcgtgtcttccgctgcgcggccatggagacccataagtattgtcattgagggtgatagcagaacaggcaaaacaatgtgggccaggtctctaggcccacataattatttatgtggacatctagacctaagcccaaaggtgtacagtaatgatgcgtggtacaacgtcattgatgacgtagacccgcattatttaaagcacttcaaggaattcatgggggcccagagggactggcaaagcaacacaaagtacgggaagcccattcaaattaaagggggaattcccactatcttcctctgcaatccaggacctacctcctcatatagggaatatctagacgaagaaaaaaacatatccttgaaaaattgggctctcaagaatgcaaccttcgtcaccctctacgagccactgttcgcaagtatcaatcaaggtccaacacaagatagccaagaagaaaccaataaggcgtaagcgtgtagacctagactgtggctgctcatactacgagcgacccactcttcaagttcatctggaacttgattaaaagaagaagaaagaaatggagaaacataaacttctaaaggaggactaaaaatcctatctaaatttgaatttaaattatgaaattgtaaaatatagtcctttggggccttttcttttaatatattgagggcctcggatttactgcctgaattgagtgctccggcgtatgcgtcgttggcagattgctgacctcctctagctgatctgccatcgatttggaaaactccaaaatcaatgaagtctccgtctttctccacgtaggtcttgacatctgttgagctcttagctgcctgaatgtttggatggaaatgtgctgacctgtttggggataccaggtcgaagaaccgttggttcttacattggtatttgccttcgaattggataagcacatggagatgtggttccccattctcgtggaattctctgcaaactttgatgtattttttatttgttggggtttctatgttttttaattgggaaagtgcttcctctttagagagagaacaattgggatatgttaggaaataattcttggcatatattttaaataaacgaggcatgttgaaatgaatcggtgtccctcaaagctctatggcaatcggtgtatcggtgtcttactaatacctcgacacctaatggcaattt

SEQ ID NO: 19 - DNA, 1188 bp, tomato yellow leaf curl virus
gccattaggtgtcgaggtattagtaagacaccgatacaccgattgccatagagctttgagggacaccgattcatttcaacatgcctcgtttatttaaaatatatgccaagaattatttcctaacatatcccaattgttctctctctaaagaggaagcactttcccaattaaaaaacatagaaaccccaacaaataaaaaatacatcaaagtttgcagagaattccacgagaatggggaaccacatctccatgtgcttatccaattcgaaggcaaataccaatgtaagaaccaacggttcttcgacctggtatccccaaacaggtcagcacatttccatccaaacattcaggcagctaagagctcaacagatgtcaagacctacgtggagaaagacggagacttcattgattttggagttttccaaatcgatggcagatcagctagaggaggtcagcaatctgccaacgacgcatacgccggagcactcaattcaggcagtaaatccgaggccctcaatatattaaaagaaaaggccccaaaggactatattttacaatttcataatttaaattcaaatttagataggatttttagtcctcctttagaagtttatgtttctccatttctttcttcttcttttaatcaagttccagatgaacttgaagagtgggtcgccgagaacgtcgtgtcttccgctgcgcggccatggagacccataagtattgtcattgagggtgatagcagaacaggcaaaacaatgtgggccaggtctctaggcccacataattatttatgtggacatctagacctaagcccaaaggtgtacagtaatgatgcgtggtacaacgtcattgatgacgtagacccgcattatttaaagcacttcaaggaattcatgggggcccagagggactggcaaagcaacacaaagtacgggaagcccattcaaattaaagggggaattcccactatcttcctctgcaatccaggacctacctcctcatatagggaatatctagacgaagaaaaaaacatatccttgaaaaattgggctctcaagaatgcaaccttcgtcaccctctacgagccactgttcgcaagtatcaatcaaggtccaacacaagatagccaagaagaaaccaataaggcgtaagcgtgtagacctagactgtggctgctcatactac

SEQ ID NO: 20 - DNA, 1190 bp, tomato yellow leaf curl virus
ccaaattgccattaggtgtcgaggtattagtaagacaccgatacaccgattgccatagagctttgagggacaccgattcatttcaacatgcctcgtttatttaaaatatatgccaagaattatttcctaacatatcccaattgttctctctctaaagaggaagcactttcccaattaaaaaacatagaaaccccaacaaataaaaaatacatcaaagtttgcagagaattccacgagaatggggaaccacatctccatgtgcttatccaattcgaaggcaaataccaatgtaagaaccaacggttcttcgacctggtatccccaaacaggtcagcacatttccatccaaacattcaggcagctaagagctcaacagatgtcaagacctacgtggagaaagacggagacttcattgattttggagttttccaaatcgatggcagatcagctagaggaggtcagcaatctgccaacgacgcatacgccggagcactcaattcaggcagtaaatccgaggccctcaatatattaaaagaaaaggccccaaaggactatattttacaatttcataatttaaattcaaatttagataggatttttagtcctcctttagaagtttatgtttctccatttctttcttcttcttttaatcaagttccagatgaacttgaagagtgggtcgccgagaacgtcgtgtcttccgctgcgcggccatggagacccataagtattgtcattgagggtgatagcagaacaggcaaaacaatgtgggccaggtctctaggcccacataattatttatgtggacatctagacctaagcccaaaggtgtacagtaatgatgcgtggtacaacgtcattgatgacgtagacccgcattatttaaagcacttcaaggaattcatgggggcccagagggactggcaaagcaacacaaagtacgggaagcccattcaaattaaagggggaattcccactatcttcctctgcaatccaggacctacctcctcatatagggaatatctagacgaagaaaaaaacatatccttgaaaaattgggctctcaagaatgcaaccttcgtcaccctctacgagccactgttcgcaagtatcaatcaaggtccaacacaagatagccaagaagaaaccaataaggcgtaagcgtgtagacctagactgtggctgctcat

SEQ ID NO: 21 - DNA, 1193 bp, tomato yellow leaf curl virus
gtagtatgagcagccacagtctaggtctacacgcttacgccttattggtttcttcttggctatcttgtgttggaccttgattgatacttgcgaacagtggctcgtagagggtgacgaaggttgcattcttgagagcccaatttttcaaggatatgtttttttcttcgtctagatattccctatatgaggaggtaggtcctggattgcagaggaagatagtgggaattccccctttaatttgaatgggcttcccgtactttgtgttgctttgccagtccctctgggcccccatgaattccttgaagtgctttaaataatgcgggtctacgtcatcaatgacgttgtaccacgcatcattactgtacacctttgggcttaggtctagatgtccacataaataattatgtgggcctagagacctggcccacattgttttgcctgttctgctatcaccctcaatgacaatacttatgggtctccatggccgcgcagcggaagacacgacgttctcggcgacccactcttcaagttcatctggaacttgattaaaagaagaagaaagaaatggagaaacataaacttctaaaggaggactaaaaatcctatctaaatttgaatttaaattatgaaattgtaaaatatagtcctttggggccttttcttttaatatattgagggcctcggatttactgcctgaattgagtgctccggcgtatgcgtcgttggcagattgctgacctcctctagctgatctgccatcgatttggaaaactccaaaatcaatgaagtctccgtctttctccacgtaggtcttgacatctgttgagctcttagctgcctgaatgtttggatggaaatgtgctgacctgtttggggataccaggtcgaagaaccgttggttcttacattggtatttgccttcgaattggataagcacatggagatgtggttccccattctcgtggaattctctgcaaactttgatgtattttttatttgttggggtttctatgttttttaattgggaaagtgcttcctctttagagagagaacaattgggatatgttaggaaataattcttggcatatattttaaataaacgaggcatgttgaaatgaatcggtgtccctcaaagctctatggcaatcggtgtatcggtgtcttactaatacctcgacacctaatggcaattt

SEQ ID NO: 22 - DNA, 676 bp, tomato yellow leaf curl virus
gccattaggtgtcgaggtattagtaagacaccgatacaccgattgccatagagctttgagggacaccgattcatttcaacatgcctcgtttatttaaaatatatgccaagaattatttcctaacatatcccaattgttctctctctaaagaggaagcactttcccaattaaaaaacatagaaaccccaacaaataaaaaatacatcaaagtttgcagagaattccacgagaatggggaaccacatctccatgtgcttatccaattcgaaggcaaataccaatgtaagaaccaacggttcttcgacctggtatccccaaacaggtcagcacatttccatccaaacattcaggcagctaagagctcaacagatgtcaagacctacgtggagaaagacggagacttcattgattttggagttttccaaatcgatggcagatcagctagaggaggtcagcaatctgccaacgacgcatacgccggagcactcaattcaggcagtaaatccgaggccctcaatatattaaaagaaaaggccccaaaggactatattttacaatttcataatttaaattcaaatttagataggatttttagtcctcctttagaagtttatgtttctccatttctttcttcttcttttaatcaagttccagatgaacttgaagagtgggtcgc

SEQ ID NO: 23 - DNA, 547 bp, tomato yellow leaf curl virus
gcgacccactcttcaagttcatctggaacttgattaaaagaagaagaaagaaatggagaaacataaacttctaaaggaggactaaaaatcctatctaaatttgaatttaaattatgaaattgtaaaatatagtcctttggggccttttcttttaatatattgagggcctcggatttactgcctgaattgagtgctccggcgtatgcgtcgttggcagattgctgacctcctctagctgatctgccatcgatttggaaaactccaaaatcaatgaagtctccgtctttctccacgtaggtcttgacatctgttgagctcttagctgcctgaatgtttggatggaaatgtgctgacctgtttggggataccaggtcgaagaaccgttggttcttacattggtatttgccttcgaattggataagcacatggagatgtggttccccattctcgtggaattctctgcaaactttgatgtattttttatttgttggggtttctatgttttttaattgggaaagtgcttcctctttagagagagaacaattgg